(19) United States
(12) Patent Application Publication
(10) Pub. No.: US 2026/0345803 A1
(43) Pub. Date: Nov. 5, 2026

(54) An Integrin Alpha10 Antibody Drug Conjugate

(71) Applicant: Targinta AB, Lund (SE)

(72) Inventors: Evy Lundgren Åkerlund, Bjärred (SE); Katarzyna Chmielarska Masoumi, Lund (SE); Xiaoli Huang, Hjärup (SE)

(73) Assignee: Targinta AB, Lund (SE)

(21) Appl. No.: 19/106,323

(22) PCT Filed: Aug. 31, 2023
(86) PCT No.: PCT/EP2023/073919

§ 371 Date: Feb. 25, 2025

(30) Foreign Application Priority Data
Aug. 31, 2022 (EP) 22193257.7

(51) Int. Cl. A61K 47/68; A61K 39/00; A61P 35/00; C07K 16/28

(52) U.S. Cl. CPC A61K 47/6809; A61K 47/6849; A61K 47/6889; A61P 35/00; C07K 16/2842; A61K 2039/505; C07K 2317/24; C07K 2317/565; C07K 2317/73; C07K 2317/77; C07K 2317/92; C07K 2317/94

(57) ABSTRACT

The present invention relates to an integrin alpha10 antibody drug conjugate and medical uses thereof.

### TECHNICAL FIELD

The present invention relates to an integrin alpha10 antibody drug conjugate and medical uses thereof.

### BACKGROUND

Treatment of aggressive cancers are often restricted to surgery, radiation or chemotherapy. The latter is associated with poor efficacy and high toxicity. Antibody-drug conjugates (ADCs) allows for tumour-selective drug delivery that may improve efficacy and reduce off-target toxicity [1]. The ADC approach builds on a potent cytotoxic agent, a so called “payload”, conjugated to a tumour-targeting antibody. Upon binding to its antigen on the tumour cell surface, the majority of ADCs follow a similar mode of action that involves internalization and subsequent payload release.

This typically kills the target cell, and sometimes also nearby cells, which is referred to as the bystander effect. This could be beneficial for treatment of solid tumours where there is often heterogeneity in terms of expression of tumour-associated antigens [2]. Integrin alpha10 (gene name ITGA10) is a cell surface protein that belongs to the collagen-binding integrin subfamily, which consists of integrin alpha1beta1, alpha2beta1, alpha10beta1, and alpha11beta1 [3]. Sequence analysis shows that the integrin alpha10 subunit has the highest identity with integrin alpha 11 (43%) and an identity of 33 and 31% with integrin alpha1 and alpha2, respectively. Integrin alpha10beta1 is normally expressed on chondrocytes in articular cartilage, in the vertebral column, in trachea and in the cartilage supporting the bronchi and on some cells in specialised fibrous tissues such as periosteum and perichondrium that likely represent mesenchymal stem cells [3-7]. However, integrin alpha10beta1 is highly expressed in various aggressive cancers such as triple negative breast cancer, glioblastoma, prostate cancer, pancreatic cancer and lung cancer [8,9] (WO 2020/212416). Expression of integrin alpha10beta1 is associated with metastasis and increased tumour aggressiveness (WO 2020/212416). High expression of integrin alpha10beta1 correlates with poor prognosis in several types of cancers including glioblastoma [8]. The restricted expression in normal tissues and high expression in cancer tissues makes integrin alpha10beta1 a promising target for the development of ADCs.

### SUMMARY

In one aspect, the present disclosure relates to an antibody or antigen-binding fragment thereof with binding specificity for integrin alpha10, wherein the antibody or antigen-binding fragment comprises:
a light chain variable region comprising
   a) a CDR-L1 comprising or consisting of an amino acid sequence of SEQ ID NO: 1;
   b) a CDR-L2 comprising or consisting of an amino acid sequence of SEQ ID NO: 2; and
   c) a CDR-L3 comprising or consisting of an amino acid sequence of SEQ ID NO: 3;
   and
   a heavy chain variable region comprising
   d) a CDR-H1 comprising or consisting of an amino acid sequence selected from the group consisting of SEQ ID NO: 4, SEQ ID NO:5, SEQ ID NO:6 and SEQ ID NO: 7;
   e) a CDR-H2 comprising or consisting of an amino acid sequence of SEQ ID NO: 8; and
   f) a CDR-H3 comprising or consisting of an amino acid sequence of SEQ ID NO: 9.

Another aspect of the present disclosure provides a polynucleotide encoding an antibody or antigen-binding fragment thereof according to the present disclosure or a component polypeptide chain thereof.

Another aspect of the present disclosure provides a vector comprising a polynucleotide disclosed herein.

Another aspect of the present disclosure provides a recombinant host cell comprising a polynucleotide disclosed herein.

Another aspect of the present disclosure provides a method for producing an antibody or antigen-binding fragment thereof according to the present disclosure, the method comprising culturing a host cell comprising the polynucleotide or the vector according to the present disclosure, under conditions which permit expression of the encoded antibody or antigen-binding fragment thereof.

Another aspect of the present disclosure provides for an in vitro method for the detection of cells expressing integrin alpha10beta1 in a subject, the method comprising:
a) providing a sample of cells from a subject to be tested, such as biopsy tissue or blood sample;
b) optionally, extracting and/or purifying the cells present in the sample;
c) contacting an antibody or antigen-binding fragment thereof disclosed herein with cells present in the sample;
d) determining whether the antibody or antigen-binding fragment thereof binds to the cells
wherein the binding of the antibody or antigen-binding fragment thereof to the cells is indicative of the presence of a disease or disorder associated with cells expression integrin alpha10 in the tissue of a subject.

Another aspect of the present disclosure provides for an in vitro method for identifying a patient with a disease or disorder associated with cells expressing integrin alpha10 who would benefit from treatment with an antibody or antigen-binding fragment thereof according to the present disclosure, the method comprising:
a) providing a sample, such as biopsy tissue or blood sample from a patient to be tested;
b) optionally, extracting and/or purifying the cells present in the sample;
c) contacting an antibody or antigen-binding fragment thereof according to the present disclosure with the sample;
d) determining whether the antibody or antigen-binding fragment thereof binds to an integrin alpha10 subunit in the sample,
   wherein the binding of the antibody or antigen-binding fragment thereof to an integrin alpha10 subunit is indicative of a patient who would benefit from treatment with an antibody or antigen-binding fragment thereof according to the present disclosure.

Another aspect of the present disclosure provides for an in vitro method the detection of cells expressing integrin alpha10, the method comprising:
a) contacting an antibody or antigen-binding fragment thereof according to the present disclosure with cells to be analysed for their expression of integrin alpha10;
b) determining whether the antibody or antigen-binding fragment thereof binds to the cells
   wherein the binding of the antibody or antigen-binding fragment thereof to the cells is indicative of the presence of a disease or disorder associated with cells expression integrin alpha10 in the tissue of a subject.

Another aspect of the present disclosure provides for in vivo imaging the expression of the integrin alpha10beta1 in a mammal, the method comprising the steps of
a) Providing a mammal,
b) Providing an antibody or antigen-binding fragment thereof according to the present disclosure,
c) administering the antibody or antigen-binding fragment thereof according to the present disclosure to the mammal so as to allow the antibody or a fragment thereof to bind to an extracellular domain of integrin alpha10beta1 of cells in said mammal,
d) optionally adding a second labelled antibody or a fragment thereof to the sample, wherein the second antibody or a fragment thereof binds to the antibody or a fragment thereof in c),
e) detecting the antibody or antigen-binding fragment thereof according to the present disclosure of said cells in c), or optionally detecting the second labelled antibody or a fragment thereof in d) bound to the antibody or a fragment thereof, and
f) creating an image of the detected antibody or a fragment thereof, thereby imaging the expression of integrin alpha10beta1 on cells in a mammal in vivo.

In one aspect, the present disclosure provides for an antibody-drug conjugate directed against integrin alpha10 comprising:
a) an antibody or antigen-binding fragment thereof according to the present disclosure,
b) an active agent, and
c) optionally, a linker which links a) to b).

In another aspect, the present disclosure provides a pharmaceutical composition comprising: the antibody or antigen-binding fragment thereof or the antibody-drug conjugate according to the present disclosure, and a pharmaceutically acceptable buffer, diluent, carrier, adjuvant or excipient.

In another aspect, the present disclosure provides a method for delivery of an active agent to a cell expressing alpha10beta1 comprising administering to said cell the antibody-drug conjugate or the pharmaceutical composition according to the present disclosure, such that the active agent is delivered to said cell.

In another aspect, the present disclosure provides for the antibody-drug conjugate as described herein, for use as a medicament.

In another aspect, the present disclosure provides for the antibody-drug conjugate as described herein, for use in treating a patient with a disease or disorder associated with cells expressing integrin alpha10.

In another aspect, the present disclosure provides for the antibody-drug conjugate as described herein, for use in the treatment of a neoplastic disease or disorder.

In another aspect, the present disclosure provides a method for treatment of a disease characterized by expression of integrin alpha10beta1 in a subject, comprising administering to the subject the antibody-drug conjugate according to or the pharmaceutical composition according to the present disclosure.

In another aspect, the present disclosure provides a use of the antibody-drug conjugate or the pharmaceutical composition according to the present disclosure for treatment of a disease.

In another aspect, the present disclosure provides a kit comprising the antibody-drug conjugate or the pharmaceutical composition according to the present disclosure optionally further comprising means for administering the antibody-drug conjugate to a subject and/or instructions for use.

### DESCRIPTION OF DRAWINGS

FIG. 1: bis-mPEG-Glu-(Val-cit-PAB-DMEDA-PNU159682)-6′-amino-β-cyclodextrin. The structure shows the conjugation technology, the linker and payload used in the antibody drug conjugates of the present disclosure. The structure is described more in detail in the section “Antibody-drug conjugate” herein. The curvy line indicates the attachment with an antibody of the present disclosure.

FIG. 2: Internalization of five lead humanized antibody variants. Internalization of five antibody leads (Th-Ab9, Th-Ab11, Th-Ab12, Th-Ab14 and Th-Ab15) as well as a chimeric antibody (Th-Ab0) and the mouse antibody (TM-Ab) was performed in C2C12alpha10 cells. The figure shows the percentage of antibody internalization after 4 hours incubation at 37 °C.

FIG. 3: Binding specificity and affinity of Th-Ab12-ADC to integrin alpha10. Binding specificity of Th-Ab12-ADC and the isotype control ADC (Ctrl-ADC) to C2C12alpha10 and C2C12alpha11 cells were investigated by flow cytometry (A). Binding affinity to C2C12alpha10 cells (B), triple negative breast cancer cell lines BT549 (C) and Hs578T (D), and glioblastoma cell lines U3046MG (E) and U3054MG (F), rhabdoid tumour A204 (G) and osteosarcoma SJSA-1 (H) was investigated by flow cytometry. The cells were incubated with the ADCs at the indicated concentrations. The mean fluorescent intensity (MFI) represents the binding ability of the ADCs to the target at different concentrations. The affinity constant (Kd) defined as the equilibrium concentration of labelled ligand that occupies 50% of receptor sites in the absence of competition was calculated based on the dose dependent binding curve. The smaller the Kd value, the greater the binding affinity of the ligand for its target.

FIG. 4: In vitro cytotoxicity of Th-Ab12-ADC. Cytotoxicity of Th-Ab12-ADC in comparison with Ctrl-ADC and payload only (PNU-159682) was studied in C2C12alpha10 cells (A), C2C12alpha11 cells in monolayer culture (B), triple negative breast cancer cell lines BT549 (C) and Hs578T (D) cultured as sphere, glioblastoma cell lines U3046MG (E) and U3054MG (F) cultured as sphere, rhabdoid tumour A204 (G) cultured as monolayer, and osteosarcoma SJSA-1 (H) cultured as sphere. The cells were treated at the indicated concentrations for 5 days (C2C12alpha10, C2C12alpha11, A204, SJSA-1 cells) or 10 days (breast cancer and glioblastoma cells). The level of cytotoxicity was measured by WST-1 assay.

FIG. 5: Internalization of Th-Ab12-ADC. Internalization of Th-Ab12-ADC and unconjugated antibody Th-Ab12 was performed in C2C12alpha10, triple negative breast cancer cells (BT549 and Hs578T), and glioblastoma cells (U3054MG and U3046MG). The figure shows the percentage of internalization after 90 min and after 4 hours of incubation at 37 °C, compared to internalization at 4 °C.

FIG. 6: Effect of Th-Ab12-ADC on cell cycle distribution. C2C12alpha10 cells were treated with Th-Ab12-ADC or Ctrl-ADC at 0.02 nM for 5 days and the distribution (%) of the four indicated cell cycle stages was analysed using propidium iodide staining and flow cytometry.

FIG. 7: Bystander toxic effect of Th-Ab12-ADC.

Bystander effect of Th-Ab12-ADC compared to Ctrl-ADC in C2C12alpha10/C2C12alpha11 co-cultures (A). In this experiment the C2C12alpha11 cells act as bystander cells since they do not express integrin alpha10 and Th-Ab12-ADC does not cross-react with integrin alpha11. The mixed cell cultures were treated with Th-Ab12-ADC or Ctrl-ADC at 0.02 nM for 5 days. The fraction of the two different cell lines present in the co-culture after 5 days was determined by flow cytometry. B. Single cultures of C2C12alpha10 and C2C12alpha11 cells incubated with Th-Ab12-ADC at a concentration of 0.02 nM, demonstrate the specificity of the antibody for the C2C12alpha10 cells as shown by a reduction in cell number.

FIG. 8: In vivo efficacy of Th-Ab12-ADC. NUDE-NMRI mice (n=5) were subcutaneously inoculated with U3046MG patient-derived glioblastoma xenografts. Five weeks after inoculation, mice were treated with a single intravenous dose of Th-Ab12-ADC or Ctrl-ADC (black arrow), either at 1.5 mg/kg (A and B) or at 0.75 mg/kg (C and D), or with PBS. Figures A and C show that Th-Ab12-ADC reduced tumour growth compared to Ctrl-ADC and PBS, measured as reduction of tumour volume, measured by calliper. Figures B and D show that neither of the treatments had a negative effect on body weight.

FIG. 9. Competition assay with Th-Ab12 versus Tm-Ab. C2C12a10 cells were incubated with primary antibody against integrin α10 (Th-Ab12 or Tm-Ab) at a concentration of 10 μg/ml. After 30 min incubation with primary antibody, cells were washed twice with FACS buffer. Secondary antibody was then added according to Table 3 and incubated for 30 min. After incubation cells were washed twice with FACS buffer. Finally, a third antibody was added to samples 4 and 5 for the last incubation according to Table 3. Figures A and B show that in C2C12a10 cells, when Th-Ab12 antibody was incubated first (sample 4; 4 on X-axis), Th-Ab12 showed the same binding degree as the single staining (sample 2; 2 on X-axis) while Tm-Ab did not show any binding (sample 4; 4 on X-axis). When Tm-Ab was incubated first (sample 5; 5 on X-axis), addition of Th-Ab12 caused the binding signal for Tm-Ab to decrease of 20% and Th-Ab12 bound to 40% (FIG. 9B).

FIG. 10. Exemplary view of an antibody-drug conjugate according to the present disclosure (Ab=antibody; Conj. Unit=conjugating unit; Func. Unit=functional unit).

### DETAILED DESCRIPTION

### Definitions

As used herein, the singular forms “a”, “an” and “the” include plural referents unless the context clearly states otherwise. Thus, for example, reference to “an antibody” includes a plurality of such antibodies. Similarly, “an anti-integrin alpha10 antibody” can also refer to “Anti-integrin alpha10 antibodies”, as for example the antibody variants described in Examples 1 to 8.

As used herein, the term “some embodiments” can include one, or more than one embodiment.

“Integrin alpha10” or “Integrin alpha10 subunit” or “Integrin alpha10 polypeptide” as used herein refers to the alpha10 subunit of the heterodimeric protein integrin alpha10beta1. This denotation does not exclude the presence of the beta1 subunit bound to the alpha10 subunit thus forming the integrin alpha10beta1 heterodimer. “Alpha” and “a”, as well as “alpha10” and “alpha10” are equivalent terms. “Integrin alpha10” as used herein may also refer to the polynucleotide transcript encoding the alpha10 subunit of the heterodimeric protein integrin alpha10beta1, and fragments thereof.

“Anti-integrin alpha10 antibody” or “Integrin alpha10 antibody” or “Anti-integrin alpha10 subunit antibody” as used herein refers to an antibody capable of recognizing and binding to at least the alpha10 integrin of the heterodimeric protein integrin alpha10beta1. These antibodies may be antibodies that recognize an epitope of the heterodimeric protein integrin alpha10beta1, wherein the epitope comprises amino acid residues of both the alpha10 and the beta1 integrin polypeptides.

As used herein, “the antibody or antigen-binding fragment of the invention/disclosure” can be referred to as “the polypeptide of the invention/disclosure” or “the antibody polypeptide, or antigen-binding fragment thereof”, since an antibody and fragments thereof are polypeptides.

As used herein, the term “an antibody or an antigen-binding fragment thereof”, includes substantially intact antibodies as well as fragments and derivatives of antibodies. An intact antibody can be regarded as an antibody comprising variable light regions, variable heavy regions, constant light regions and constant heavy regions. It further includes chimeric antibodies, humanized antibodies, isolated human antibodies, single chain antibodies, bispecific antibodies, antibody heavy chains, antibody light chains, homodimers and heterodimers of antibody heavy and/or light chains, and antigen-binding fragments and derivatives of the same. Suitable antigen-binding fragments and derivatives include, but are not necessarily limited to, Fv fragments (e.g. single chain Fv and disulphide-bonded Fv), Fab-like fragments (e.g. Fab fragments, Fab′ fragments and F (ab) 2 fragments), single variable domains (e.g. VH and VL domains) and domain antibodies (dAbs, including single and dual formats [i.e. dAb-linker-dAb]). The potential advantages of using antibody fragments, rather than whole antibodies, are several-fold. The smaller size of the fragments may lead to improved pharmacological properties, such as better penetration of solid tissue. Moreover, antigen-binding fragments such as Fab, Fv, ScFv and dAb antibody fragments can be expressed in and secreted from E. coli, thus allowing the facile production of large amounts of the said fragments.

As used herein, the term the “antibody” or “antigen-binding fragment of the invention/disclosure” is also intended to encompass antibody mimics (for example, non-antibody scaffold structures that have a high degree of stability yet allow variability to be introduced at certain positions). Those skilled in the art of biochemistry will be familiar with many such molecules, as discussed in Gebauer & Skerra, 2009, Curr Opin Chem Biol 13 (3): 245-255 (the disclosures of which are incorporated herein by reference). Exemplary antibody mimics include: affibodies (also called Trinectins; Nygren, 2008, FEBS J, 275, 2668-2676); CTLDs (also called Tetranectins; Innovations Pharmac. Technol. (2006), 27-30); adnectins (also called monobodies; Meth. Mol. Biol., 352 (2007), 95-109); anticalins (Drug Discovery Today (2005), 10, 23-33); DARPins (ankyrins; Nat. Biotechnol. (2004), 22, 575-582); avimers (Nat. Biotechnol. (2005), 23, 1556-1561); microbodies (FEBS J, (2007), 274, 86-95); peptide aptamers (Expert. Opin. Biol. Ther. (2005), 5, 783-797); Kunitz domains (J. Pharmacol. Exp. Ther. (2006) 318, 803-809); affilins (Trends. Biotechnol. (2005), 23, 514-522); affimers (Avacta Life Sciences, Wetherby, UK).

The term “amino acid” as used herein includes the standard twenty genetically-encoded amino acids and their corresponding stereoisomers in the ‘D’ form (as compared to the natural ‘L’ form), omega-amino acids and other naturally-occurring amino acids, unconventional amino acids (e.g. α,α-disubstituted amino acids, N-alkyl amino acids, etc.) and chemically derivatised amino acids as described herein. When an amino acid is being specifically enumerated, such as “alanine” or “Ala” or “A”, the term refers to both L-alanine and D-alanine unless explicitly stated otherwise. Other unconventional amino acids may also be suitable components for polypeptides (the antibody or antigen-binding fragment thereof) of the present disclosure, as long as the desired functional property is retained by the antibody or antigen-binding fragment. For the amino acid sequences shown, each encoded amino acid residue, where appropriate, is represented by a single letter designation, corresponding to the trivial name of the conventional amino acid.

As used herein “expression vector” or “vector” refers to a DNA construct containing a DNA sequence that is operably linked to a suitable control sequence capable of effecting the expression of the DNA in a suitable host. Such control sequences may, e.g., include a promoter to effect transcription, an optional operator sequence to control such transcription, a sequence encoding suitable mRNA ribosome binding sites and sequences which control termination of transcription and translation. The vector may, e.g., be a plasmid, a phage or simply a potential genomic insert. Once transformed into a suitable host, the vector may, e.g., replicate and function independently of the host genome, or may, in some instances, integrate into the genome itself. Expression vectors are designed, for example, as described in Li et al. (Construction strategies for developing expression vectors for recombinant monoclonal antibody production in CHO cells, Mol Biol Rep. 2018 December; 45 (6): 2907-2912.)

“Subject” as used herein denotes a mammal, such as a rodent, a feline, a canine, an equine and a primate. Preferably a subject according to the disclosure is a human.

A “sample” as used herein encompasses any subject and a variety of sample types obtained from any subject. Examples of samples useful in the disclosed methods include but are not limited to a subject, a liquid tissue sample such as blood, or a solid tissue sample such as biopsy material or tissue cultures or cells derived there from and the progeny thereof. For example, biological samples include cells obtained from a tissue sample collected from a subject. Thus, samples encompass clinical samples, cells in culture, cell supernatants, cell lysates, and tissue samples, e.g. tissue samples from breast tissue, lung tissue, prostate tissue, pancreatic tissue, bone tissue, cartilage tissue, fat tissue, muscle tissue and connective tissue.

A “cancer” as used herein refers to any malignant and/or invasive growth or tumor caused by abnormal cell growth. As used herein “cancer” refers to tumors named for the type of cells that form them. A cancer or tumor comprise of tumor cells or cancer cells. A cancer or tumor comprises also the cancer or tumor microenvironment, which can also comprise MSCs, fibroblasts, endothelial cells, pericytes, adipocytes, immune cells, tumor associated macrophages TAMs. Part of a cancer or tumor might be stroma cells, for example connective tissue cells such as fibroblasts. Examples of solid tumors include but are not limited to sarcomas and carcinomas. The term “cancer” includes but is not limited to a primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of different type from latter one. Cancer, tumor, neoplasm are used herein as synonyms.

“Detection”, “detect”, “detecting” as used herein includes qualitative and/or quantitative detection (measuring levels) with or without reference to a control, and further refers to the identification of the presence, absence or quantity of a given target, specifically the target of an integrin alpha10 subunit.

By “disorder associated with cells expressing integrin alpha10beta1” we include such diseases or disorders wherein the pathological cells which are responsible, directly or indirectly, for the disorder express integrin alpha10beta1 on the cell surface. It will be appreciated that the cells expressing integrin alpha10beta1 may be immune cells, cells of the connective tissue such as fibroblasts or neoplastic cells (cancer cells), e.g. tumor cells, per se. In addition, such cells include pathological stem cells (i.e. cancer stem cells, or CSCs) and progenitor cells which are responsible, directly or indirectly, for the development of a neoplastic disease or disorder in an individual. Examples of CSCs are disclosed in Visvader & Lindeman, 2008, Nat Rev Cancer 8:755-768, the disclosures of which are incorporated herein by reference.

Alternatively, or in addition, the cells expressing integrin alpha10beta1 may be associated indirectly with the neoplastic disease or disorder, for example, they may mediate cellular processes required for the cells to survive.

### Integrin Alpha 10 Antibody

In one aspect, the present disclosure relates to an antibody or antigen-binding fragment thereof with binding specificity for integrin alpha10, wherein the antibody or antigen-binding fragment comprises:
a light chain variable region comprising
   a) a CDR-L1 comprising or consisting of an amino acid sequence of SEQ ID NO: 1;
   b) a CDR-L2 comprising or consisting of an amino acid sequence of SEQ ID NO: 2; and
   c) a CDR-L3 comprising or consisting of an amino acid sequence of SEQ ID NO: 3;
      and/or
      a heavy chain variable region comprising
   d) a CDR-H1 comprising or consisting of an amino acid sequence selected from the group consisting of SEQ ID NO: 4, SEQ ID NO:5, SEQ ID NO:6 and SEQ ID NO: 7;
   e) a CDR-H2 comprising or consisting of an amino acid sequence of SEQ ID NO: 8; and
   f) a CDR-H3 comprising or consisting of an amino acid sequence of SEQ ID NO: 9.

In one aspect, the present disclosure relates to an antibody or antigen-binding fragment thereof with binding specificity for integrin alpha10, wherein the antibody or antigen-binding fragment comprises:
a light chain variable region comprising
   a) a CDR-L1 comprising or consisting of an amino acid sequence of SEQ ID NO: 1;
   b) a CDR-L2 comprising or consisting of an amino acid sequence of SEQ ID NO: 2; and
   c) a CDR-L3 comprising or consisting of an amino acid sequence of SEQ ID NO: 3;
      and
      a heavy chain variable region comprising
   d) a CDR-H1 comprising or consisting of an amino acid sequence selected from the group consisting of SEQ ID NO: 4, SEQ ID NO:5, SEQ ID NO:6 and SEQ ID NO: 7;
   e) a CDR-H2 comprising or consisting of an amino acid sequence of SEQ ID NO: 8; and
   f) a CDR-H3 comprising or consisting of an amino acid sequence of SEQ ID NO: 9.

The antibody or antigen-binding fragment of the disclosure has specificity for the alpha10 subunit in the integrin alpha10beta1. By “specificity” we mean that the antibody or antigen-binding fragment is capable of binding to integrin alpha10beta1 in vivo, i.e., under the physiological conditions in which integrin alpha10beta1 exists within the human body. Preferably, the antibody or antigen-binding fragment does not bind or has only minor binding to any other protein in vivo. Alternatively, it is meant that the antibody or antigen-binding fragment is capable of binding to integrin alpha10beta1 ex vivo or in vitro. Such binding specificity may be determined by methods well known in the art, such as ELISA, immunohistochemistry, immunoprecipitation, Western blots and flow cytometry using transfected cells expressing integrin alpha10beta1. Advantageously, the antibody or antigen-binding fragment is capable of binding selectively to integrin alpha10beta1, i.e., it binds at least 10-fold more strongly to integrin alpha10beta1 than to any other proteins.

In some embodiments, the antibody or antigen-binding fragment thereof comprises:
a) an immunoglobulin light chain variable region comprising or consisting of the amino acid sequence selected from the group consisting of SEQ ID NO: 10, SEQ ID NO: 11 and SEQ ID NO:12 or a sequence having at least 85% sequence identity to any of SEQ ID NO: 10, SEQ ID NO: 11 and SEQ ID NO: 12, such as at least 95%, such as 98% or 99% sequence identity to any of SEQ ID NO: 10, SEQ ID NO: 11 and SEQ ID NO: 12; and/or
b) an immunoglobulin heavy chain variable region comprising or consisting of the amino acid sequence selected from the group consisting of SEQ ID NO: 13, SEQ ID NO: 14, SEQ ID NO:15, SEQ ID NO: 16, SEQ ID NO: 17, SEQ ID NO: 18 and SEQ ID NO:19 or a sequence having at least 85% sequence identity to any of SEQ ID NO: 13, SEQ ID NO: 14, SEQ ID NO: 15, SEQ ID NO: 16, SEQ ID NO: 17, SEQ ID NO: 18 and SEQ ID NO: 19, such as at least 95%, such as 98% or 99% sequence identity to any of SEQ ID NO: 13, SEQ ID NO:14, SEQ ID NO: 15, SEQ ID NO: 16, SEQ ID NO: 17, SEQ ID NO: 18 and SEQ ID NO: 19.

In some embodiments, the antibody or antigen-binding fragment thereof comprises:
a) an immunoglobulin light chain variable region comprising or consisting of the amino acid sequence of SEQ ID NO: 12 and an immunoglobulin heavy chain variable region comprising or consisting of the amino acid sequence of SEQ ID NO: 16; or
b) an immunoglobulin light chain variable region comprising or consisting of the amino acid sequence of SEQ ID NO: 12 and an immunoglobulin heavy chain variable region comprising or consisting of the amino acid sequence of SEQ ID NO: 15; or
c) an immunoglobulin light chain variable region comprising or consisting of the amino acid sequence of SEQ ID NO: 11 and an immunoglobulin heavy chain variable region comprising or consisting of the amino acid sequence of SEQ ID NO: 16; or
d) an immunoglobulin light chain variable region comprising or consisting of the amino acid sequence of SEQ ID NO: 11 and an immunoglobulin heavy chain variable region comprising or consisting of the amino acid sequence of SEQ ID NO: 17; or
e) an immunoglobulin light chain variable region comprising or consisting of the amino acid sequence of SEQ ID NO: 12 and an immunoglobulin heavy chain variable region comprising or consisting of the amino acid sequence of SEQ ID NO: 17.

In some embodiments, the antibody or antigen-binding fragment thereof comprises:
a) an immunoglobulin light chain variable region comprising CDR-L1 of SEQ ID NO:1, CDR-L2 of SEQ ID NO:2 and CDR-L3 of SEQ ID NO:3;
   and
b) an immunoglobulin heavy chain variable region comprising
   i. CDR-H1 of SEQ ID NO:4, CDR-H2 of SEQ ID NO:8 and CDR-H3 of SEQ ID NO:9, or
   ii. CDR-H1 of SEQ ID NO:5, CDR-H2 of SEQ ID NO:8 and CDR-H3 of SEQ ID NO:9.

In some embodiments, the antibody or antigen-binding fragment thereof comprises:
a) an immunoglobulin light chain variable region comprising or consisting of the amino acid sequence of SEQ ID NO: 12; and/or
b) an immunoglobulin heavy chain variable region comprising or consisting of the amino acid sequence of SEQ ID NO: 16.

In some embodiments, the antibody or antigen-binding fragment thereof comprises:
a light chain variable region comprising
   a) a CDR-L1 comprising or consisting of an amino acid sequence of SEQ ID NO: 1;
   b) a CDR-L2 comprising or consisting of an amino acid sequence of SEQ ID NO: 2; and
   c) a CDR-L3 comprising or consisting of an amino acid sequence of SEQ ID NO: 3;
and/or
a heavy chain variable region comprising
   d) a CDR-H1 comprising or consisting of an amino acid sequence of SEQ ID NO: 4;
   e) a CDR-H2 comprising or consisting of an amino acid sequence of SEQ ID NO: 8; and
   f) a CDR-H3 comprising or consisting of an amino acid sequence of SEQ ID NO: 9.

In some embodiments, the antibody or antigen-binding fragment thereof comprises an IgG light chain constant region and an IgG heavy chain constant region.

In some embodiments, the antibody or antigen-binding fragment thereof comprises:
a) an immunoglobulin light chain constant region comprising or consisting of the amino acid sequence of SEQ ID NO: 20; and/or
b) an immunoglobulin heavy chain constant region comprising or consisting of the amino acid sequence of SEQ ID NO: 21.

In some embodiments, the antibody or antigen-binding fragment thereof with binding specificity for the integrin alpha10 polypeptide, which is a part of an integrin alpha10beta1 heterodimer.

In some embodiments, the antibody or antigen-binding fragment thereof with binding specificity for the integrin alpha10beta1 which is human integrin alpha10beta1.

In some embodiments, the antibody or antigen-binding fragment thereof with binding specificity for the integrin alpha10beta1 which is expressed on the surface of a cell.

In some embodiments, the antibody or antigen-binding fragment thereof binds to the extracellular I-domain of the integrin alpha10 subunit.

In some embodiments of the present disclosure, the antibody or antigen-binding fragment thereof with binding specificity for the integrin alpha10beta1 is selected from a mouse antibody, a chimeric antibody, a human antibody, a humanised antibody, a humanised antigen-binding fragment, a Fab fragment, a Fab′ fragment, an F(ab′)2 fragment, an Fv, a single chain antibody (SCA) such as an scFv, a disulphide-bonded Fv, the variable portion of the heavy and/or light chains thereof, and a Fab miniantibody.

In some embodiments, the antibody or antigen-binding fragment thereof with binding specificity for the integrin alpha10beta1 is a monoclonal antibody or antigen-binding fragment thereof.

In some embodiments, the antibody or antigen-binding fragment thereof with binding specificity for the integrin alpha10beta1 is a humanised or fully human monoclonal antibody or antigen-binding fragment thereof.

In some embodiments, the antibody or antigen-binding fragment thereof with binding specificity for the integrin alpha10beta1 is a recombinant antibody or antigen-binding fragment thereof.

In some embodiments, as discussed above, the antibody or antigen-binding fragment of the invention comprises or consists of an antibody mimic selected from the group comprising or consisting of affibodies, tetranectins (CTLDs), adnectins (monobodies), anticalins, DARPins (ankyrins), avimers, iMabs, microbodies, peptide aptamers, Kunitz domains and affilins.

Persons skilled in the art will further appreciate that the invention also encompasses modified versions of antibodies and antigen-binding fragments thereof, whether existing now or in the future, e.g., modified by the covalent attachment of polyethylene glycol or another suitable polymer.

In some embodiments, the antibody or antigen-binding fragment thereof with binding specificity for the integrin alpha10beta1 is conjugated to an additional moiety.

In some embodiments, the additional moiety comprises a detectable moiety, such as a detectable moiety selected from the group consisting of a fluorophore, an enzyme and a radioactive tracer or radioisotope. Thus, the antibody of the present disclosure may be useful in methods of detection of cells expressing integrin alpha10 as well as detection and diagnosis of tumours characterized by high integrin alpha10 expression, as described herein.

In some embodiments, the radioisotope is selected from the group consisting of 99mTc, 111 In, 67Ga, 68Ga, 72As, 89Zr, 1231 and 201TI.

In some embodiments, the antibody or antigen-binding fragment thereof with binding specificity for integrin alpha10 comprises a pair of detectable and cytotoxic radioisotopes, such as 86Y/90Y or 1241/211At.

In some embodiments, the antibody or antigen-binding fragment thereof with binding specificity for integrin alpha10 comprises a radioisotope which is capable of simultaneously acting in a multi-modal manner as a detectable moiety and as a cytotoxic moiety.

In some embodiments, the detectable moiety comprises or consists of a paramagnetic isotope.

In some embodiments, the paramagnetic isotope is selected from the group consisting of 157Gd, 55Mn, 162Dy, 52Cr and 56Fe.

In some embodiments, the detectable moiety is detectable by an imaging technique such as SPECT, PET, MRI, optical or ultrasound imaging.

In some embodiments, the detectable moiety is joined to the antibody or antigen-binding fragment thereof indirectly, via a linking moiety.

In some embodiments, the linking moiety is a chelator.

In some embodiments, the chelator is selected from the group consisting of derivatives of 1,4,7,10-tetraazacyclododecane-1,4,7,10, tetraacetic acid (DOTA), deferoxamine (DFO), derivatives of diethylenetriaminepentaacetic avid (DTPA), derivatives of S-2-(4-Isothiocyanatobenzyl)-1,4,7-triazacyclononane-1,4,7-triacetic acid (NOTA) and derivatives of 1,4,8,11-tetraazacyclodocedan-1,4,8,11-tetraacetic acid (TETA).

In some embodiments, the antibody or antigen-binding fragment thereof with binding specificity for integrin alpha10 does not comprise a detectable moiety.

### Integrin Alpha10 Polypeptide

Integrins are heterodimers consisting of an alpha and a beta polypeptide. The integrin alpha10beta1 heterodimer may be detected by integrin alpha10-specific antibodies and integrin alpha10 binding peptides and proteins.

In some embodiments, the integrin alpha10 polypeptide is a part of an integrin alpha10beta1 heterodimer.

In some embodiments, the integrin alpha10 polypeptide is expressed on the surface of the cells.

The integrin alpha10beta1 was originally identified as a collagen type II binding receptor on chondrocytes in 1998 (Camper et al., 1998). In vitro studies have demonstrated its binding to other collagen subtypes and to laminin (Lundgren-Åkerlund Book chapter and Thoren et al). Immunohistochemical analysis during development and in adult tissues has demonstrated a restricted localization to cartilage-containing tissues and to some fibrous tissues (Camper et al. 1998, Camper et al., 2001). Knockout mice lacking the marker have disorganized growth plates, decreased collagen in the matrix and shorter long-bones, further supporting its cell structural importance (Bengtsson et al., 2005). The amino acid sequence, variants, isoforms and sequence annotations can be found in Uniprot accession no O75578 (ITA10_HUMAN).

Integrin alpha10beta1 receptors transmit, upon binding to the extracellular ligand, intracellular signals that promote cell adhesion, migration, survival, proliferation, tumor growth and metastasis. Inhibition of the receptor thus impedes adhesion, migration, survival, proliferation, tumor growth and metastasis. This may be important for the treatment of many proliferative diseases such as cancer and inflammatory diseases.

In some embodiments, integrin alpha10 is a naturally occurring variant of integrin alpha10 polypeptide, an isoform of integrin alpha10 polypeptide or a splice variant of an integrin alpha10 polypeptide.

Integrin alpha10 can also be detected on nucleotide level by analyzing a sample for the presence of e.g. mRNA transcripts which upon translation generates an integrin alpha10 antigen as defined herein above.

### CDRs

The antibody of the present invention is defined by its characteristic complementarity-determining region (CDR) sequences. There are several approaches for defining the CDR sequences of an antibody. The CDRs of the antibody of the present invention have been defined using definition according to Kabat.

The person skilled in the art will appreciate that a set of 6 CDRs (CDR-L1, CDR-L2, CDR-L3, CDR-H1, CDR-H2, CDR-H3) may be defined according to Kabat.

Further, the person skilled in the art will appreciate that it is possible to define the CDRs of the antibody of the invention by other approaches, for example by definition of CDRs according to Chothia (Al-Lazikani et al., (1997) JMB 273, 927-948), Martin (Enhanced Chotia), Gelfand or Honneger. Further approaches exist, such as the AbM definition (combination of Kabat definition and Chothia definition used by Oxford Molecular's AbM antibody modelling software) or the contact definition (based on analysis of crystal structures). See, e.g., Kabat et al. (Sequences of Proteins of Immunological Interest, 1987 and 1991, NIH, Bethesda, Md.), Lefranc et al. (IMGT unique numbering for immunoglobulin and T cell receptor constant domains and Ig superfamily C-like domains, Dev Comp Immunol. 2005; 29 (3): 185-203) and Dondelinger et al. (Understanding the Significance and Implications of Antibody Numbering and Antigen-Binding Surface/Residue Definition, Front. Immunol., 16 Oct. 2018).

The person skilled in the art could, when provided with the Kabat CDRs as presented herein, use known information to list other CDR naming conventions or approaches (like Chothia). Thus, all CDR naming conventions or approaches are encompassed.

In certain cases, it can be beneficial to define the CDRs according to one numbering system, such as Kabat. Often, these CDR sequences are short (shorter than, for example, an approach combining numbering systems), thus providing the core sequences critical for binding. In other cases, it can be beneficial to use a combination of, for example, IMGT and Kabat CDR sequences.

However, the person skilled in the art will appreciate that a low level of mutation (typically, just one or two amino acids) within a CDR sequence may be tolerated without loss of the specificity of the antibody or antigen-binding fragment for integrin alpha10.

In some embodiments, the disclosure relates to an antibody or antigen-binding fragment thereof with binding specificity for integrin alpha10, wherein the antibody or antigen-binding fragment comprises:
a light chain variable region comprising
   a) a CDR-L1 comprising or consisting of an amino acid sequence of SEQ ID NO: 1;
   b) a CDR-L2 comprising or consisting of an amino acid sequence of SEQ ID NO: 2; and
   c) a CDR-L3 comprising or consisting of an amino acid sequence of SEQ ID NO: 3;
and/or
a heavy chain variable region comprising
   d) a CDR-H1 comprising or consisting of an amino acid sequence selected from the group consisting of SEQ ID NO: 4, SEQ ID NO:5, SEQ ID NO:6 and SEQ ID NO: 7;
   e) a CDR-H2 comprising or consisting of an amino acid sequence of SEQ ID NO: 8; and
   f) a CDR-H3 comprising or consisting of an amino acid sequence of SEQ ID NO: 9.

In some embodiments, the antibody or antigen-binding fragment thereof comprises: a light chain variable region comprising
a) a CDR-L1 comprising or consisting of an amino acid sequence of SEQ ID NO: 1;
b) a CDR-L2 comprising or consisting of an amino acid sequence of SEQ ID NO: 2; and
c) a CDR-L3 comprising or consisting of an amino acid sequence of SEQ ID NO: 3;
   and/or
   a heavy chain variable region comprising
d) a CDR-H1 comprising or consisting of an amino acid sequence of SEQ ID NO:
e) a CDR-H2 comprising or consisting of an amino acid sequence of SEQ ID NO: 8; and
f) a CDR-H3 comprising or consisting of an amino acid sequence of SEQ ID NO: 9.

In some embodiments, the antibody or antigen-binding fragment thereof comprises: a light chain variable region comprising
a) a CDR-L1 comprising or consisting of an amino acid sequence of SEQ ID NO: 1;
b) a CDR-L2 comprising or consisting of an amino acid sequence of SEQ ID NO: 2; and
c) a CDR-L3 comprising or consisting of an amino acid sequence of SEQ ID NO: 3;
   and/or
   a heavy chain variable region comprising
d) a CDR-H1 comprising or consisting of an amino acid sequence of SEQ ID NO: 5;
e) a CDR-H2 comprising or consisting of an amino acid sequence of SEQ ID NO: 8; and
f) a CDR-H3 comprising or consisting of an amino acid sequence of SEQ ID NO: 9.

In some embodiments, the antibody or antigen-binding fragment thereof with binding specificity for integrin alpha10 comprises CDRs as described above (comprising or consisting of the amino acid sequence selected from the group consisting of SEQ ID NOs 1 to 9), wherein any one of the amino acids of the CDRs has been altered for another amino acid, for example, with the proviso that no more than 2 amino acids have been so altered, such as 1 amino acid.

### Light and Heavy Chain Variable Regions

In some embodiments, the antibody or antigen-binding fragment thereof comprises:
a) an immunoglobulin light chain variable region comprising or consisting of the amino acid sequence selected from the group consisting of SEQ ID NO: 10, SEQ ID NO: 11 and SEQ ID NO: 12 or a sequence having at least 85% sequence identity to any of SEQ ID NO: 10, SEQ ID NO: 11 and SEQ ID NO:12, such as at least 95%, such as 98% or 99% sequence identity to any of SEQ ID NO: 10, SEQ ID NO: 11 and SEQ ID NO: 12; and/or
b) an immunoglobulin heavy chain variable region comprising or consisting of the amino acid sequence selected from the group consisting of SEQ ID NO: 13, SEQ ID NO: 14, SEQ ID NO: 15, SEQ ID NO: 16, SEQ ID NO: 17, SEQ ID NO: 18 and SEQ ID NO: 19 or a sequence having at least 85% sequence identity to any of SEQ ID NO: 13, SEQ ID NO: 14, SEQ ID NO: 15, SEQ ID NO: 16, SEQ ID NO: 17, SEQ ID NO: 18 and SEQ ID NO: 19, such as at least 95%, such as 98% or 99% sequence identity to any of SEQ ID NO: 13, SEQ ID NO: 14, SEQ ID NO: 15, SEQ ID NO: 16, SEQ ID NO: 17, SEQ ID NO: 18 and SEQ ID NO: 19.

In some embodiments, the antibody or antigen-binding fragment thereof comprises:
a) an immunoglobulin light chain variable region comprising or consisting of the amino acid sequence of SEQ ID NO: 12; and/or
b) an immunoglobulin heavy chain variable region comprising or consisting of the amino acid sequence of SEQ ID NO: 16.

In some embodiments, the antibody or antigen-binding fragment thereof with binding specificity for integrin alpha10 comprises a light chain variable region comprising or consisting of
a) the amino acid sequence of SEQ ID NO: 12, or an amino acid sequence having at least 85% sequence identity to SEQ ID NO: 12, for example at least 90%, 95%, 98% or 99% sequence identity; or
b) an amino acid sequence selected from the group consisting of SEQ ID NO: 10 and SEQ ID NO: 11, or an amino acid sequence having at least 85% sequence identity to SEQ ID NO: 10 and SEQ ID NO: 11, for example at least 90%, 95%, 98% or 99% sequence identity to any one of SEQ ID NO: 10 and SEQ ID NO: 11.

In some embodiments, the antibody or antigen-binding fragment thereof with binding specificity for integrin alpha10 comprises a light chain variable region comprising or consisting of the amino acid sequence of SEQ ID NO: 12, or an amino acid sequence having at least 85% sequence identity to SEQ ID NO: 12, for example at least 90%, 95%, 98% or 99% sequence identity.

In some embodiments, the antibody or antigen-binding fragment thereof with binding specificity for integrin alpha10 comprises a heavy chain variable region comprising or consisting of
a) the amino acid sequence of SEQ ID NO: 16; or an amino acid sequence having at least 85% sequence identity to SEQ ID NO: 16 for example at least 90%, 95%, 98% or 99% sequence identity; or
b) an amino acid sequence selected from the group consisting of SEQ ID NO: 13, SEQ ID NO: 14, SEQ ID NO: 15, SEQ ID NO: 17, SEQ ID NO: 18 and SEQ ID NO: 19; or an amino acid sequence having at least 85% sequence identity to SEQ ID NO: 13, SEQ ID NO: 14, SEQ ID NO: 15, SEQ ID NO: 17, SEQ ID NO: 18 and SEQ ID NO: 19, for example at least 90%, 95%, 98% or 99% sequence identity to any one of SEQ ID NO: 13, SEQ ID NO: 14, SEQ ID NO: 15, SEQ ID NO: 17, SEQ ID NO: 18 and SEQ ID NO: 19.

In some embodiments, the antibody or antigen-binding fragment thereof with binding specificity for integrin alpha10 comprises a heavy chain variable region comprising or consisting of the amino acid sequence of SEQ ID NO: 16; or an amino acid sequence having at least 85% sequence identity to SEQ ID NO: 16 for example at least 90%, 95%, 98% or 99% sequence identity.

Percent identity (or sequence identity) can be determined by, for example, the LALIGN program at the Expasy facility site (http://www.ch.embnet.org/software/LALIGN_form.html) using as parameters the global alignment option, scoring matrix BLOSUM62, opening gap penalty—14, extending gap penalty—4. Alternatively, the percent sequence identity between two polypeptides, such as parts of an antibody, may be determined using suitable computer programs, for example the GAP program of the University of Wisconsin Genetic Computing Group and it will be appreciated that percent identity is calculated in relation to polypeptides whose sequence has been aligned optimally.

The alignment may alternatively be carried out using the Clustal W program. The parameters used may be as follows:
Fast pair-wise alignment parameters: K-tuple (word) size; 1, window size; 5, gap penalty; 3, number of top diagonals; 5. Scoring method: x percent.
Multiple alignment parameters: gap open penalty; 10, gap extension penalty; 0.05.
Scoring matrix: BLOSUM.

Alternatively, the BESTFIT program may be used to determine local sequence alignments.

The person skilled in the art will consider further alterations to the above described light chain and heavy chain variable regions, for example to further optimize the antibody or antigen-binding fragment. Usually, the person skilled in the art will, as done during humanization and de-immunization procedures, consider to alter amino acids in the framework regions, i.e., outside the epitope binding CDR regions, thereby usually not altering the CDR regions.

In some embodiments, the antibody or antigen-binding fragment thereof with binding specificity for integrin alpha10 comprises a light chain variable region and/or a heavy variable chain region as described above, wherein any one of the amino acids of the framework region of the light chain variable region and/or a heavy variable chain region has been altered for another amino acid, with the proviso that no more than 5 amino acids have been so altered, such as 4 amino acids, no more than 3 amino acids, such as 2 amino acids or no more than 1 amino acid.

### Combination of Variable Light and Variable Heavy Chains

The person skilled in the art will appreciate that any of the above described variants of light chain variable regions can be combined with any of the above described variants of heavy chain variable regions.

In some embodiments, the antibody or antigen-binding fragment thereof comprises:
a) an immunoglobulin light chain variable region comprising or consisting of the amino acid sequence of SEQ ID NO: 12; and/or
b) an immunoglobulin heavy chain variable region comprising or consisting of the amino acid sequence of SEQ ID NO: 16.

These combinations of light and heavy chain variable regions are, for example, part of the humanized antibody variants as described in Example 1.

### Production of the Antibody

Another aspect of the disclosure relates to a polynucleotide encoding the antibody or antigen-binding fragment of the present disclosure, or a component polypeptide chain thereof.

By “polynucleotide” we include DNA (e.g. genomic DNA or complementary DNA) and mRNA molecules, which may be single- or double-stranded.

In some embodiments, the polynucleotide is an isolated polynucleotide.

In some embodiments, the polynucleotide is a cDNA molecule.

It will be appreciated by persons skilled in the art that the polynucleotide may be codon-optimised for expression of the antibody or antigen-binding fragment in a particular host cell, e.g. for expression in human cells (for example, see Angov, 2011, Biotechnol. J. 6 (6): 650-659, the disclosures of which are incorporated herein by reference).

In some embodiments, the polynucleotide encoding the antibody or antigen-binding fragment of the disclosure is encoding an antibody light chain or variable region thereof.

In some embodiments, the polynucleotide encoding the antibody or antigen-binding fragment of the disclosure is encoding an antibody heavy chain or variable region thereof.

In some embodiments, the polynucleotide encoding the antibody or antigen-binding fragment of the disclosure is encoding an antibody comprising:
a) an immunoglobulin light chain variable region comprising or consisting of the amino acid sequence of SEQ ID NO: 12; and/or
b) an immunoglobulin heavy chain variable region comprising or consisting of the amino acid sequence of SEQ ID NO: 16.

Another aspect of the disclosure relates to a vector comprising the polynucleotide according to another aspect of the disclosure.

In some embodiments, the vector is an expression vector.

The term “expression vector” is defined herein as a DNA molecule, for example linear or circular, which comprises a polynucleotide encoding a polypeptide of the present invention (the antibody or antigen-binding fragment thereof) and is operably linked to additional nucleotides that provide for its expression. The term “plasmid”, “expression vector” and “vector” are used interchangeably as the plasmid is the most commonly used form of vector at present. However the invention is intended to include such other forms of expression vectors that serve equivalent functions.

Another aspect of the disclosure relates to a recombinant host cell comprising the polynucleotide according to another aspect of the disclosure or a vector according to another aspect of the disclosure.

In some embodiments, the recombinant host cell is a bacterial cell.

In some embodiments, the recombinant host cell is a yeast cell.

In some embodiments, the recombinant host cell is a mammalian cell.

In some embodiments, the recombinant host cell is a human cell.

Another aspect of the disclosure relates to a method for producing the antibody or antigen-binding fragment of the another aspect of the invention, the method comprising culturing the host cell of another aspect of the disclosure comprising the polynucleotide of another aspect of the disclosure or the vector of the third aspect of the disclosure, under conditions which permit expression of the encoded antibody or antigen-binding fragment thereof.

### Detection of Integrin Alpha10

Another aspect of the disclosure provides for an in vitro method for the detection of cells expressing integrin alpha10beta1 in a subject, the method comprising:
a) providing a sample of cells from a subject to be tested, such as biopsy tissue or blood sample;
b) optionally, extracting and/or purifying the cells present in the sample;
c) contacting the antibody or antigen-binding fragment of the present disclosure with cells present in the sample;
d) determining whether the antibody or antigen-binding fragment thereof binds to the cells,
   wherein the binding of the antibody or antigen-binding fragment thereof to the cells is indicative of the presence of a disease or disorder associated with cells expression integrin alpha10 in the tissue of a subject.

Yet another aspect of the present disclosure provides for an in vitro method for identifying a patient with a disease or disorder associated with cells expressing integrin alpha10 who would benefit from treatment with an antibody or antigen-binding fragment thereof according to the present disclosure, the method comprising:
a) providing a sample, such as biopsy tissue or blood sample from a patient to be tested;
b) optionally, extracting and/or purifying the cells present in the sample;
c) contacting an antibody or antigen-binding fragment thereof according to the present disclosure with the sample;
d) determining whether the antibody or antigen-binding fragment thereof binds to an integrin alpha10 subunit in the sample,
   wherein the binding of the antibody or antigen-binding fragment thereof to an integrin alpha10 subunit is indicative of a patient who would benefit from treatment with an antibody or antigen-binding fragment thereof according to the present disclosure. The antibody or antigen-binding fragment thereof according to the present disclosure can be used to detect an integrin alpha10 subunit as found on the surface of cells, in particular cancer cells, expressing integrin alpha10 as well as freely circulating in the blood of a patient. In fact, integrin alpha10 can be released from the cell membrane, for example due to the action of proteases, and end up in blood (shedding of integrin alpha10 subunit). As a consequence of this phenomenon, the antibody or antigen-binding fragment thereof according to the present disclosure can be used to detect an integrin alpha10 subunit directly in blood of a patient.

In another aspect, the present disclosure provides for a method for the detection of cells expressing integrin alpha10, the method comprising:
a) contacting an antibody or antigen-binding fragment thereof according to the present disclosure with cells to be analysed for their expression of integrin alpha10;
b) determining whether the antibody or antigen-binding fragment thereof binds to the cells
   wherein the binding of the antibody or antigen-binding fragment thereof to the cells is indicative of the presence of a disease or disorder associated with cells expression integrin alpha10 in the tissue of a subject.

In some embodiments, said method can be is an in vivo method or an in vitro method.

In another aspect, the disclosure provides for a method for in vivo imaging the expression of the integrin alpha10beta1 in a mammal, the method comprising the steps of
a) Providing a mammal,
b) Providing an antibody or antigen-binding fragment thereof according to the present disclosure,
c) administering the antibody or antigen-binding fragment thereof according to the present disclosure to the mammal so as to allow the antibody or a fragment thereof to bind to an extracellular domain of integrin alpha10beta1 of cells in said mammal,
d) optionally adding a second labelled antibody or a fragment thereof to the sample, wherein the second antibody or a fragment thereof binds to the antibody or a fragment thereof in c),
e) detecting the antibody or antigen-binding fragment thereof according to the present disclosure of said cells in c), or optionally detecting the second labelled antibody or a fragment thereof in d) bound to the antibody or a fragment thereof, and
f) creating an image of the detected antibody or a fragment thereof, thereby imaging the expression of integrin alpha10beta1 on cells in a mammal in vivo.

In some embodiments, the antibody is covalently bound to a detectable moiety, such as a detectable moiety selected from the group consisting of a fluorophore, an enzyme, a radioactive tracer or a radioisotope. The integrin alpha10 antigen may also be detected by detecting a peptide, protein or polypeptide other than integrin alpha10 polypeptide, wherein said other peptide, protein or polypeptide is capable of specifically binding to an integrin alpha10 antigen. In some embodiments said peptide, protein or polypeptide is linked to an enzyme, a fluorophore or a radioactive tracer. The radioactive tracer may e.g. be selected from a positron emitter, or a gamma emitter. Conjugation of the antibody to a detectable moiety facilitates and improves detection of said antibody, which in turn may facilitate detection of integrin alpha10-expressing cells in a sample and so the diagnosis of a cancer.

In some embodiments, the antibody of the present disclosure can be used to detect integrin alpha10 on cells, in tissues, in blood of a sample obtained from a mammal, such as in vitro, or even in vivo and/or in situ by using in vivo antibody-based detection techniques described herein and/or known to the person of skill in art.

The person of skill in the art is capable of selecting the standard laboratory equipment for detection of the integrin alpha10 antibodies, depending on the situation and physical state of the sample.

In some embodiments, the person of skill in the art would conduct the detection step using flow cytometry such as Fluorescence-Activated Cell Sorting (FACS).

Typical immunological methods well known in the art include but are not limited to western blot, enzyme linked immunosorbent assay (ELISA), radioimmunoassay (RIA), immunohistochemistry (IHC), immunofluorescent assay (IF), fluorescence in situ hybridization (FISH).

Detecting integrin alpha10 can be achieved using methods well known in the art of detection and imaging, such as clinical imaging, such as conventional fluorescence microscopes, confocal microscope, 2-photon microscopes, stimulated emission depletion (STED) etc.

In some embodiments, the detectable moiety is selected from the group consisting of a fluorophore, an enzyme or a radioactive tracer.

Typical methods for detection of cell surface antigens in vivo are well known in the art include but are not limited to fluorescence imaging, positron emission tomography, x-ray computed tomography (CT), magnetic resonance imaging (MRI) and functional magnetic resonance imaging (fMRI), ultrasound and single-photon emission computed tomography (SPECT). In particular cell surface antigens can be imaged in vivo using immunolabelling with a radioactive tracer bound to an antibody or other specifically binding protein.

In some embodiments, the antibodies used for in vivo imaging are antibody fragments such as Fab fragments, and single chain antibodies due to their smaller size and absence of effector function.

### Antibody-Drug Conjugate

An antibody-drug conjugate (ADC) is formed by covalent biochemical conjugation of a monoclonal antibody with high toxic payload via a small molecular linker. ADC manufacturing is a multiple step process that can be divided into three distinct stages: cGMP production of the antibody, cGMP synthesis of the drug-linker complex, and conjugation to form an ADC. The conjugated antibodies subsequently undergo an extensive purification process.

In one aspect, the present disclosure provides for an antibody-drug conjugate directed against integrin alpha10 comprising:
a) an antibody or antigen-binding fragment thereof as defined in the present disclosure,
b) an active agent, and
c) optionally, a linker which links a) to b).

In some embodiments of the present disclosure, the antibody is attached to the linker through a conjugating unit.

In some embodiments, the antibody-drug conjugate of the present disclosure also comprises a functional unit, for example a unit that improved solubility of the antibody-drug conjugate. FIG. 10 is an exemplary view of an antibody-drug conjugate according to the present disclosure (Ab=antibody; Conj. Unit=conjugating unit; Func. Unit=functional unit). The various parts of the antibody-drug conjugate of the present disclosure are described more in detail herein.

### Active Agent

The antibody drug conjugate (ADC) of the present disclosure comprises an active agent, i.e. a drug, which can be delivered intracellularly to cells expressing integrin alpha10beta1.

The active agent may e.g. be a therapeutic agent, a cytotoxic agent, a radioisotope or a detectable label. In a preferred embodiment the active agent is a therapeutic agent.

In some embodiments said active agent is a therapeutic agent, a cytotoxic agent, a microtubule toxin or a transcription toxin.

In some embodiments the active agent is a chemotherapeutic agent. Classes of chemotherapeutic agents include alkylating agents, anthracyclines, antimetabolites, anti-microtubule/anti-mitotic agents, histone deacetylase inhibitors, kinase inhibitors, dihydrofolate reductase inhibitors, peptide antibiotics, platinum-based antineoplastics, topoisomerase inhibitors and cytotoxic antibiotics.

In some embodiments the active agent may be or comprise a radioisotope. The radioisotope may serve as a radiation emitter either for treatment of affected tissues or for diagnostic purposes. In one embodiment, the radioisotope may consist of or comprise 60Co, 89Sr, 90Y, 99mTc, 131I, 137Cs, 153Sm, or 223Rd. In one embodiment of the present disclosure, the radioisotope may be in combination with a chelator such as DOTA or EDTA or others which are well known in the art.

In some embodiments the active agent is a therapeutic agent. Classes of therapeutic agents include DNA crosslinking agents, DNA alkylating agents, DNA strand scission agents, anthracyclines, antimetabolites, anti-microtubule/anti-mitotic agents, histone deacetylase inhibitors, kinase inhibitors, metabolism inhibitors, peptide antibiotics, immune checkpoint inhibitors, platinum-based antineoplastics, topoisomerase inhibitors, DNA or RNA polymerase inhibitors, immunomodulators, nucleotide-based agents, and cytotoxic antibiotics.

In some embodiments, the active agent is a cytotoxic agent.

In some embodiments, the active agent is a therapeutic agent, such as a therapeutic agent selected from the group consisting of microtubule toxins, DNA toxins and transcription toxins.

In some embodiments, the active agent is a microtubule toxin, such as a microtubule toxin selected from the group consisting of Auristatin-based toxins, Maytansinoid-based toxins, Tubulysins-based toxins and Eribulin.

In some embodiments, the active agent is a transcription toxin, such as an RNA polymerase II and/or III inhibiting agent.

In some embodiments, the active agent is a therapeutic agent, such as a therapeutic agent selected from the group consisting of alkylating agents, anthracyclines, antimetabolites, anti-microtubule/anti-mitotic agents, histone deacetylase inhibitors, kinase inhibitors, peptide antibiotics, platinum-based antineoplastics, topoisomerase inhibitors and cytotoxic antibiotics.

In some embodiments, the active agent is a transcription toxin selected from the group consisting of Doxorubicin, Doxorubicin derivatives and Amanitin.

In some embodiments, the active agent is an anthracycline, such as an anthracycline selected from Daunorubicin, doxorubicin, epirubicin, idarubicin, and 3′-deamino-3″-4′-anhydro-[2″(S)-methoxy-3″(R)-hydroxy-4″-morpholinyl]doxorubicin (PNU159682).

In a preferred embodiment, the active agent is 3′-deamino-3″-4′-anhydro-[2″(S)-methoxy-3″(R)-hydroxy-4″-morpholinyl]doxorubicin (PNU159682), which is represented in Formula X:

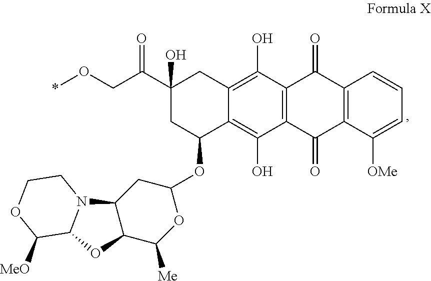 [SMILES: *OCC(=O)[C@]1(O)Cc2c(O)c3c(c(O)c2[C@@H](OC2C[C@H]4[C@H](O[C@@H]5[C@@H](OC)OCCN54)[C@H](C)O2)C1)C(=O)c1c(OC)cccc1C3=O]

wherein * indicates an attachment site to a linker.

In one embodiment, the active agent is a DNA- or RNA-polymerase inhibitor, such as a polymerase inhibitor selected from amanitin or alpha-amanitin or derivatives thereof, actinomycin D, and aphidicolin.

In one embodiment, the active agent is a nucleotide-based agent, such as an RNA- or DNA-oligonucleotide, such as a siRNA or a miRNA

There may be one or more units of drug per antibody molecule. The ratio between the number of drug molecules per antibody is denoted the drug-to-antibody ratio (DAR). In one embodiment, the DAR is between 1 and 10, such as between 2 and 8, for example between 3 and 6, such as 1, 2, 3 or 4. Preferably, the DAR is 1 or 2.

### Linker

A stable link between the antibody and the active agent is an important aspect of ADC technology. Linkers may e.g. be based on chemical motifs including disulfides, hydrazones or peptides (cleavable), or thioethers (non-cleavable), and control the distribution and delivery of the cytotoxic agent to the target cell. Cleavable and non-cleavable types of linkers have been proven to be safe in preclinical and clinical trials.

In a preferred embodiment of the present disclosure, the ADC as disclosed herein comprises a linker that links the antibody to the active agent.

In some embodiments, the antibody-drug conjugate comprises a linker selected from a cleavable and a non-cleavable linker.

The type of linker, cleavable or non-cleavable, lends specific properties to the delivered drug. For example, cleavable linkers can e.g. be cleaved by enzymes in the target cell, leading to efficient intracellular release of the active agent, for example a cytotoxic agent. In contrast, an ADC containing a non-cleavable linker has no mechanism for drug release, and must rely on mechanisms such as degradation of the targeting antibody, for drug release. Furthermore, as is appreciated by those skilled in the art, the linker composition may influence critical factors such as solubility and pharmacokinetic properties of the ADC as a whole.

For both types of linker, drug release is crucial for obtaining a cellular effect. Drugs which are able to freely diffuse across cell membranes may escape from the targeted cell and, in a process called “bystander killing,” also attack neighbouring cells, such as cancer cells in the vicinity of the integrin alpha10beta1 expressing target cell. Cleavable groups include a disulfide bond, an amide bond, a substituted amide bond in the form of a peptide bond, a thioamide, bond, an ester bond, a thioester bond, a vicinal diol bond, or a hemiacetal. These, or other cleavable bonds, may include enzymatically-cleavable bonds, such as peptide bonds (cleaved by peptidases), phosphate bonds (cleaved by phosphatases), nucleic acid bonds (cleaved by endonucleases), and sugar bonds (cleaved by glycosidases).

In a further embodiment of the present disclosure, the linker is a cleavable linker allowing for intracellular release of the active agent inside the target cells.

The person of skill in the art is capable of selecting a suitable linker from those routinely used for ADCs. Examples of commonly used linkers include but not limited to Val-Cit-PAB, Fmoc-Val-Cit-PAB, Fmoc-Val-Cit-PAB-PNP, MC-Val-Cit-PAB-PNP, Phe-Lys (Trt)-PAB, Fmoc-Phe-Lys (Trt)-PAB, Fmoc-Phe-Lys (Trt)-PAB-PNP, Ala-Ala-Asn-PAB TFA salt, Fmoc-Ala-Ala-Asn-PAB-PNP, Fmoc-Gly3-Val-Cit-PAB, Fmoc-Gly3-Val-Cit-PAB-PNP, SMCC, Py-ds-Prp-OSu, Py-ds-dmBut-OSu, Py-ds-dmBut-OPFP, Py-ds-Prp-OPFP, MAL-HA-OSu, MAL-di-EG-OPFP, MAL-tri-EG-OPFP, MAL-tetra-EG-OPFP, N3-di-EG-OPFP, N3-tri-EG-OPFP, N3-tetra-EG-OPFP, ALD-BZ-OSu, ALD-di-EG-OSu, ALD-tetra-EG-OSu, ALD-di-EG-OPFP, ALD-tetra-EG-OPFP, PHA-di-EG-OPFP, PHA-tetra-EG-OPFP.

In some embodiments, the linker is an enzyme-cleavable linker, such as a cathepsin cleavable linker.

In some embodiments, the linker comprises a peptide linker. The choice of peptide sequence is critical to the success of the conjugate. In some embodiments the linker is stable to serum proteases, yet is cleaved by lysosomal enzymes in the target cell.

In some embodiments, the linker is an enzyme-cleavable peptide-containing linker, such as a cathepsin cleavable peptide-containing linker. Cathepsin can be one of several cathepsin types, being one of a group of lysosomal proteases.

In some embodiments, the linker comprises a dipeptide, such as Valine-Citrulline (Val-Cit) or Valine-Alanine (Val-Ala).

In some embodiments, the linker comprises the dipeptide Valine-Citrulline (Val-Cit, Formula V).

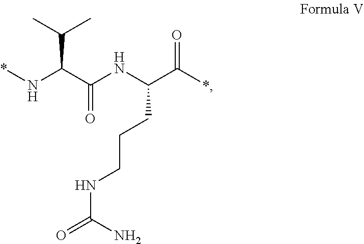

wherein * indicates an attachment site to other parts of a linker and/or to a conjugating unit and/or to an active agent.

Enzymatically cleavable linkers may include a “self-immolative molecule” also referred to as “self-immolative spacer” or “self-immolative linker” to spatially separate the drug from the site of enzymatic cleavage. The direct attachment of a drug to a peptide linker can result in proteolytic release of an amino acid adduct of the drug, thereby impairing its activity. The use of a self-immolative spacer allows for the elimination of the fully active, chemically unmodified drug upon amide bond hydrolysis.

Thus, in some embodiments, the linker comprises one or more self-immolative molecules. Examples of self-immolative molecules include p-aminobenzylcarbamoyl (PAB) and N,N′-Dimethylethylenediamine (DMEDA).

In some embodiments, the linker comprises a dipeptide and p-aminobenzylcarbamoyl (PAB, Formula P).

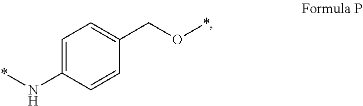

wherein * indicates an attachment site to other parts of a linker and/or to a conjugating unit and/or to an active agent.

In some embodiments, the linker comprises a N,N′-Dimethylethylenediamine (DMEDA, Formula D).

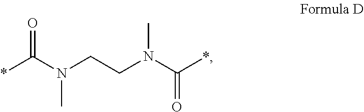

wherein * indicates an attachment site to other parts of a linker and/or to a conjugating unit and/or to an active agent.

In a preferred embodiment, the linker comprises a dipeptide, such as Valine-Citrulline (Val-Cit) or Valine-Alanine (Val-Ala), and the self-immolative molecules p-aminobenzylcarbamoyl (PAB) and N,N′-Dimethylethylenediamine (DMEDA).

In some embodiments, the linker comprises or consists of Val-Cit-PAB-DMEDA, as represented in Formula Y.

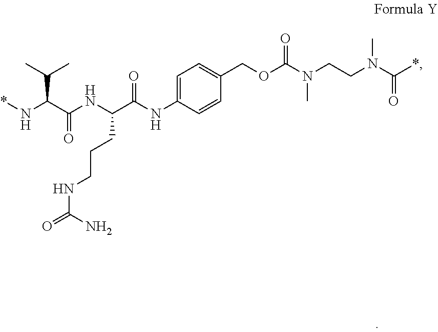

wherein * indicates an attachment site to other parts of a linker and/or to a conjugating unit and/or to an active agent.

In some embodiments, the antibody-drug conjugate of one aspect of present disclosure further comprises a conjugating unit, such as a conjugating unit derived from reactive groups such as a functionalized benzoic acid, an activated carboxylic acid derivative, an amino group, a maleimide group or derivatives thereof, N-hydroxysuccinimide, bis-sulfones, azides and alkynes via click chemistry, reactive attachment groups directed to modified or unmodified protein-bound carbohydrate, peptide sequences that are required for enzymatic reactions, by reaction with the antibody or a chemically or enzymatically generated derivative thereof.

In some embodiments, the conjugating unit is derived from a functionalized bis-sulfone group according to Formula C below:

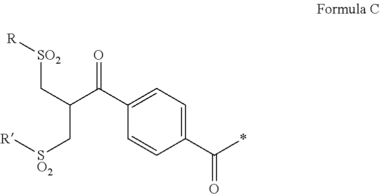

wherein * indicates an attachment site to a linker, and wherein R and R′ are each independently selected form the group consisting of selected from the group consisting of C₁-C₆ alkyl, a C₃-C₇ cycloalkyl, a C₃-C₇ heterocycloalkyl, phenyl, C5-C₁₀ aryl, each of which may optionally be substituted by one or more selected from halogen, cyano, amino, C₁-C₆ alkyl, C₁-C₆ alkoxy, phenyl, and C₅-C₁₀ aryl.

For example, the antibody or antigen-binding fragment thereof with binding specificity for integrin alpha10 disclosed herein may be conjugated to the conjugating unit via a number of inter-chain thiol bridging groups across both the light chain and heavy chain constant regions of the antibody, as well as inter-chain thiol bridging groups between the heavy chain constant regions of the antibody.

In some embodiments, when the conjugating unit is derived from a functionalized bis-sulfone group, such as Formula C or C′ or C″, the conjugation may occur by means of a disulfide reduction, such as by means of a reaction between one or more-SH groups of cysteines within the antibody or antigen-binding fragment thereof and the sulfone groups of the conjugating unit. Consequently, the SO₂-PEGn-unit may by a leaving group which detaches from the conjugated ADC.

In some embodiments, the conjugating unit is derived from a functionalized bis-sulfone group according to formula C′

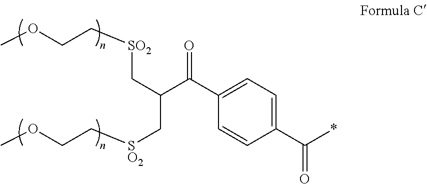

wherein * indicates an attachment site to a linker, and wherein n is an integer between 1 and 10.

In some embodiments, the conjugating unit is derived from a functionalized bis-sulfone group according to formula C″, also herein referred to as bis-mPEG.

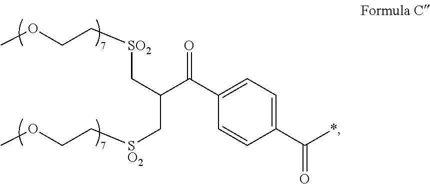

wherein * indicates an attachment site.

The conjugating unit conjugates the antibody against integrin alpha10 of the present disclosure to the linker, which is attached to the active agent.

In some embodiments, the conjugating unit is derived from a functionalized bis-sulfone group such as any of Formula C, C′ or C″, and is functionalized with a glutamic acid. For example, in some embodiments, the conjugating unit is derived from a functionalized bis-sulfone group according to formula C″, also herein referred to as bis-mPEG, and functionalized with a glutamic acid to form a bis-mPEG-Glu, wherein the Glu is attached to the * in Formula C″.

In some embodiments, the antibody-drug conjugate of one aspect of present disclosure comprises a functional unit, such as a unit that improves solubility of the antibody-drug conjugate, such as a cyclodextrin or a PEG molecule.

In some embodiments, the functional unit comprises or consists of 6′-amino-β-cyclodextrin.

In some embodiments, the functional unit comprises or consists of a PEG molecule having a molecular weight of 10 kDa or below, such as 8 kDa, such as 5 kDa or below, such as 4.6 kDA, such as 4 kDa, such as 1 kDa, such as 0.6 kDA, such as 0.4 KDa, such as 0.2 kDA.

In some embodiments, wherein the functional unit, such as the unit that improves solubility of the antibody-drug conjugate comprises or consists of a PEG molecule consisting of 72 PEG units or less, such as 50 PEG units, such as 20 PEG units.

In some embodiments the functional unit is attached to the conjugating unit.

In some embodiments the functional unit is attached to the glutamic acid comprised in the conjugating unit.

In some embodiments the functional unit is attached to the linker.

In some embodiments the antibody-drug conjugate of the present disclosure comprises bis-mPEG-Glu-(Val-cit-PAB-DMEDA-PNU159682)-6′-amino-β-cyclodextrin, as represented in Formula A.

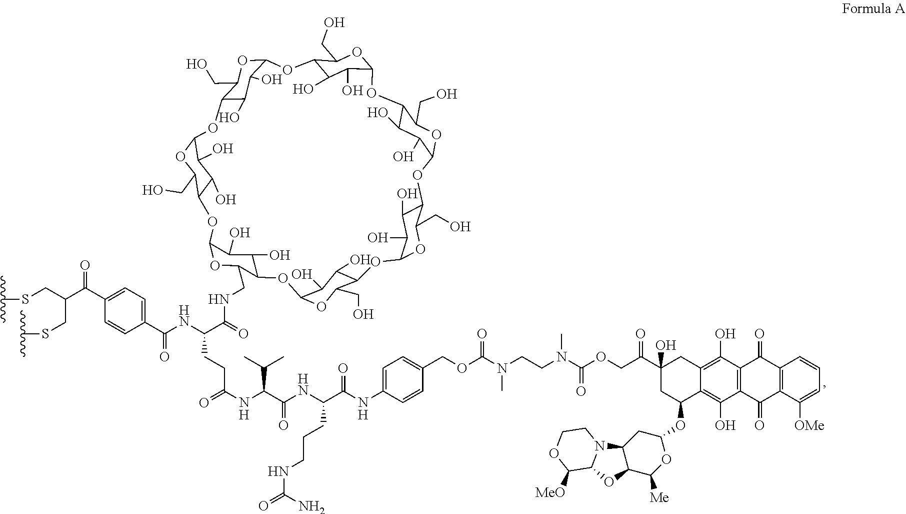 [SMILES: *SCC(CS*)C(=O)c1ccc(C(=O)N[C@@H](CCC(=O)N[C@H](C(=O)N[C@@H](CCCNC(N)=O)C(=O)Nc2ccc(COC(=O)N(C)CCN(C)C(=O)OCC(=O)[C@]3(O)Cc4c(O)c5c(c(O)c4[C@@H](O[C@H]4C[C@H]6[C@H](O[C@@H]7[C@@H](OC)OCCN76)[C@H](C)O4)C3)C(=O)c3c(OC)cccc3C5=O)cc2)C(C)C)C(=O)NCC2OC3OC4C(CO)OC(OC5C(CO)OC(OC6C(CO)OC(OC7C(CO)OC(OC8C(CO)OC(OC9C(CO)OC(OC2C(O)C3O)C(O)C9O)C(O)C8O)C(O)C7O)C(O)C6O)C(O)C5O)C(O)C4O)cc1]

wherein [symbol] indicates the attachment with the antibody.

In some embodiments the antibody-drug conjugate of the present disclosure comprises:
a) the antibody or antigen-binding fragment thereof according to another aspect of the present disclosure, wherein the antibody or antigen-binding fragment thereof comprises
   a light chain variable region comprising a CDR-L1 consisting of SEQ ID NO:1, a CDR-L2 consisting of SEQ ID NO:2 and a CDR-L3 consisting of SEQ ID NO:3; and
   a heavy chain variable region comprising a CDR-H1 consisting of any one of SEQ ID NO: 4, SEQ ID NO:5, SEQ ID NO:6 and SEQ ID NO:7, a CDR-H2 consisting of SEQ ID NO:5 and a CDR-H3 consisting of SEQ ID NO:6;
b) a conjugating unit, such as a conjugating unit derived from a bis-sulfone group according to formula C″ and functionalized with a glutamic acid (bis-mPEG-Glu);
c) a linker comprising or consisting of Val-cit-PAB-DMEDA;
d) a functional unit, such as 6′-amino-β-cyclodextrin; and
e) the active agent PNU159682.

In some embodiments the antibody-drug conjugate comprises an antibody or antigen-binding fragment that comprises: a light chain variable region comprising
a) a CDR-L1 comprising or consisting of an amino acid sequence of SEQ ID NO: 1;

b) a CDR-L2 comprising or consisting of an amino acid sequence of SEQ ID NO: 2; and

c) a CDR-L3 comprising or consisting of an amino acid sequence of SEQ ID NO: 3;

and
a heavy chain variable region comprising
d) a CDR-H1 comprising or consisting of an amino acid sequence of SEQ ID NO: 4;
e) a CDR-H2 comprising or consisting of an amino acid sequence of SEQ ID NO: 8; and
f) a CDR-H3 comprising or consisting of an amino acid sequence of SEQ ID NO: 9.

In some embodiments the antibody-drug conjugate comprises an antibody or antigen-binding fragment that comprises: a light chain variable region comprising
a) a CDR-L1 comprising or consisting of an amino acid sequence of SEQ ID NO: 1;
b) a CDR-L2 comprising or consisting of an amino acid sequence of SEQ ID NO: 2; and
c) a CDR-L3 comprising or consisting of an amino acid sequence of SEQ ID NO: 3;
and
a heavy chain variable region comprising
d) a CDR-H1 comprising or consisting of an amino acid sequence of SEQ ID NO: 5;
e) a CDR-H2 comprising or consisting of an amino acid sequence of SEQ ID NO: 8; and
f) a CDR-H3 comprising or consisting of an amino acid sequence of SEQ ID NO: 9.

In some embodiments the antibody-drug conjugate comprises an antibody or antigen-binding fragment that comprises:
a) an immunoglobulin light chain variable region comprising or consisting of the amino acid sequence of SEQ ID NO: 12 and an immunoglobulin heavy chain variable region comprising or consisting of the amino acid sequence of SEQ ID NO: 16; or
b) an immunoglobulin light chain variable region comprising or consisting of the amino acid sequence of SEQ ID NO: 12 and an immunoglobulin heavy chain variable region comprising or consisting of the amino acid sequence of SEQ ID NO: 15; or
c) an immunoglobulin light chain variable region comprising or consisting of the amino acid sequence of SEQ ID NO: 11 and an immunoglobulin heavy chain variable region comprising or consisting of the amino acid sequence of SEQ ID NO: 16; or
d) an immunoglobulin light chain variable region comprising or consisting of the amino acid sequence of SEQ ID NO: 11 and an immunoglobulin heavy chain variable region comprising or consisting of the amino acid sequence of SEQ ID NO: 17; or
e) an immunoglobulin light chain variable region comprising or consisting of the amino acid sequence of SEQ ID NO: 12 and an immunoglobulin heavy chain variable region comprising or consisting of the amino acid sequence of SEQ ID NO: 17.

In a preferred embodiment, the antibody-drug conjugate comprises an antibody or antigen-binding fragment that comprises:
a) an immunoglobulin light chain variable region comprising or consisting of the amino acid sequence of SEQ ID NO: 12; and
b) an immunoglobulin heavy chain variable region comprising or consisting of the amino acid sequence of SEQ ID NO: 16.

In a preferred embodiment, the antibody-drug conjugate of the present disclosure comprises the antibody or antigen-binding fragment thereof of the present disclosure which is linked to a conjugating unit, such as bis-mPEG of Formula C″ functionalized with a glutamic acid (bis-mPEG-Glu), which is linked to a linker comprising or consisting of Val-Cit-PAB-DMEDA, which is linked to the active agent PNU159682, wherein the conjugating unit is also linked to a functional unit, such as 6′-amino-β-cyclodextrin, via the glutamic acid comprised in the conjugating unit.

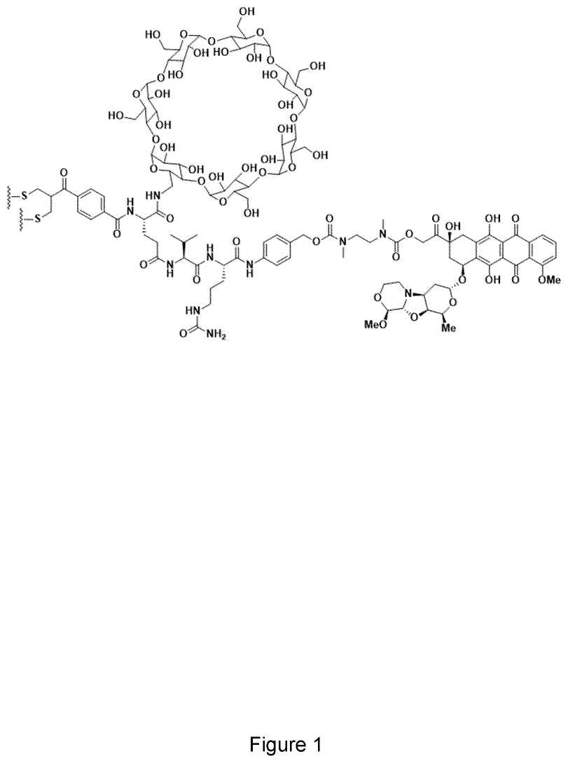

In a preferred embodiment, the antibody-drug conjugate of the present disclosure comprises the antibody or antigen-binding fragment thereof of the present disclosure which is linked to a drug conjugate as illustrated in FIG. 1.

### Therapeutic Use

The ADCs directed against integrin alpha10 as described herein are useful for the delivery of active agents, such as therapeutic or cytotoxic agents to cells expressing alpha10 beta1 and thus for the treatment of a range of diseases and disorder associated with cells expressing integrin alpha10beta1.

In one embodiment, the present disclosure provides a pharmaceutical composition comprising an effective amount of the ADC, as described herein, together with a pharmaceutically acceptable buffer, diluent, carrier, adjuvant or excipient.

The pharmaceutical compositions may be prepared in a manner known in the art that is sufficiently storage stable and suitable for administration to humans and/or animals. For example, the pharmaceutical compositions may be lyophilised, e.g. through freeze drying, spray drying, spray cooling, or through use of particle formation from supercritical particle formation.

By “pharmaceutically acceptable” we mean a non-toxic material that does not decrease the effectiveness of the anti-integrin alpha10 ADC. Such pharmaceutically acceptable buffers, carriers or excipients are well-known in the art (see Remington's Pharmaceutical Sciences, 18th edition, A. R Gennaro, Ed., Mack Publishing Company (1990) and handbook of Pharmaceutical Excipients, 3rd edition, A. Kibbe, Ed., Pharmaceutical Press (2000), the disclosures of which are incorporated herein by reference).

The term “buffer” is intended to mean an aqueous solution containing an acid-base mixture with the purpose of stabilising pH. Pharmaceutically acceptable buffers are well known in the art.

The term “diluent” is intended to mean an aqueous or non-aqueous solution with the purpose of diluting the agent in the pharmaceutical preparation.

The term “adjuvant” is intended to mean any compound added to the formulation to increase the biological effect of the agent of the invention. The adjuvant may be one or more of zinc, copper or silver salts with different anions, for example, but not limited to fluoride, chloride, bromide, iodide, thiocyanate, sulfite, hydroxide, phosphate, carbonate, lactate, glycolate, citrate, borate, tartrate, and acetates of different acyl composition. The adjuvant may also be cationic polymers such as cationic cellulose ethers, cationic cellulose esters, deacetylated hyaluronic acid, chitosan, cationic dendrimers, cationic synthetic polymers such as poly(vinyl imidazole), and cationic polypeptides such as polyhistidine, polylysine, polyarginine, and peptides containing these amino acids.

The excipient may be one or more of carbohydrates, polymers, lipids and minerals. Examples of carbohydrates include lactose, glucose, sucrose, mannitol, and cyclodextrines, which are added to the composition, e.g., for facilitating lyophilisation. Examples of polymers are starch, cellulose ethers, cellulose carboxymethylcellulose, hydroxypropylmethyl cellulose, hydroxyethyl cellulose, ethylhydroxyethyl cellulose, alginates, carageenans, hyaluronic acid and derivatives thereof, polyacrylic acid, polysulphonate, polyethylenglycol/polyethylene oxide, polyethyleneoxide/polypropylene oxide copolymers, polyvinylalcohol/polyvinylacetate of different degree of hydrolysis, and polyvinylpyrrolidone, all of different molecular weight, which are added to the composition, e.g., for viscosity control, for achieving bioadhesion, or for protecting the lipid from chemical and proteolytic degradation. Examples of lipids are fatty acids, phospholipids, mono-, di-, and triglycerides, ceramides, sphingolipids and glycolipids, all of different acyl chain length and saturation, egg lecithin, soy lecithin, hydrogenated egg and soy lecithin, which are added to the composition for reasons similar to those for polymers. Examples of minerals are talc, magnesium oxide, zinc oxide and titanium oxide, which are added to the composition to obtain benefits such as reduction of liquid accumulation or advantageous pigment properties.

The ADCs of the present disclosure may be formulated into any type of pharmaceutical composition known in the art to be suitable for the delivery thereof.

The ADCs of the present disclosure or pharmaceutical compositions comprising the ADCs may be administered via any suitable route known to those skilled in the art. Thus, possible routes of administration include parenteral (intravenous, subcutaneous, and intramuscular), topical, ocular, nasal, pulmonar, buccal, oral, vaginal and rectal. Also, administration from implants is possible.

In one preferred embodiment, the pharmaceutical compositions are administered parenterally, for example, intravenously, intracerebroventricularly, intraarticularly, intra-arterially, intraperitoneally, intrathecally, intraventricularly, intrasternally, intracranially, intramuscularly or subcutaneously, or they may be administered by infusion techniques. They are conveniently used in the form of a sterile aqueous solution which may contain other substances, for example, enough salts or glucose to make the solution isotonic with blood. The aqueous solutions should be suitably buffered if necessary. The preparation of suitable parenteral formulations under sterile conditions is readily accomplished by standard pharmaceutical techniques well known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

In one embodiment the ADCs of the present disclosure are administered in situ in the body of a subject during a surgery, to the damaged site.

In one embodiment the ADCs of the present disclosure are administered intravenously.

In one embodiment the ADCs of the present disclosure are administered subcutaneously.

In one embodiment the ADCs of the present disclosure are administered intracranially or intracerebrally.

The pharmaceutical compositions will be administered to a patient in a pharmaceutically effective amount. A ‘therapeutically effective amount’, or ‘effective amount’, or ‘therapeutically effective’, as used herein, refers to that amount which provides a therapeutic effect for a given condition and administration regimen. This is a predetermined quantity of active material calculated to produce a desired therapeutic effect in association with the required additive and diluent, i.e. a carrier or administration vehicle. Further, it is intended to mean an amount sufficient to reduce, and most preferably prevent, a clinically significant deficit in the activity, function and response of the host. Alternatively, a therapeutically effective amount is sufficient to cause an improvement in a clinically significant condition in a host. As is appreciated by those skilled in the art, the amount of a compound may vary depending on its specific activity. Suitable dosage amounts may contain a predetermined quantity of active composition calculated to produce the desired therapeutic effect in association with the required diluent. A therapeutically effective amount can be determined by the ordinarily skilled medical or veterinary worker based on patient characteristics, such as age, weight, sex, condition, complications, other diseases, etc., as is well known in the art. The administration of the pharmaceutically effective dose can be carried out both by single administration in the form of an individual dose unit, or else several smaller dose units, and also by multiple administrations of subdivided doses at specific intervals. Alternatively, the dose may be provided as a continuous infusion over a prolonged period.

It will be appreciated by persons skilled in the art that the ADCs targeting integrin alpha10beta1 described herein may be administered alone or in combination with other therapeutic agents. For example, the ADCs targeting integrin alpha10 described herein may be administered in combination with a range of anti-cancer agents, such as antimetabolites, alkylating agents, anthracyclines and other cytotoxic antibiotics, vinca alkyloids, anti-microtubule/anti-mitotic agents, histone deacetylase inhibitors, kinase inhibitors, peptide antibiotics, platinum-based antineoplastics, etoposide, taxanes, topoisomerase inhibitors, antiproliferative immunosuppressants, corticosteroids, sex hormones and hormone antagonists, cytotoxic antibiotics and other therapeutic agents.

In one embodiment the ADC of the present disclosure is administered in conjunction with additional reagents and/or therapeutics that may increase the functional efficiency of the ADC, such as established or novel drugs that increase lysosomal membrane permeability, thereby facilitating molecular entry from the lysosome interior to the cytoplasm, or drugs that increase the permeability of the blood-brain barrier.

In one aspect the present disclosure provides for a pharmaceutical composition comprising the antibody or antigen-binding fragment thereof according to another aspect of the disclosure, or the antibody-drug conjugate according to the present disclosure, and a pharmaceutically acceptable buffer, diluent, carrier, adjuvant or excipient.

In another aspect, the present disclosure provides for a method for delivery an active agent to a cell expressing alpha10beta1 comprising administering to said cell the antibody-drug conjugate as described herein, or the pharmaceutical composition according to another aspect of the disclosure, such that the active agent is delivered to said cell.

In another aspect, the present disclosure provides for the antibody-drug conjugate or the pharmaceutical composition as described herein, for use as a medicament.

In another aspect, the present disclosure provides for the antibody-drug conjugate or the pharmaceutical composition as described herein, for use in treating a patient with a disease or disorder associated with cells expressing integrin alpha10.

In some embodiments, the cells expressing integrin alpha10 are malignant cells or tumor-associated cells, such as cancer associated fibroblast (CAFs), stromal cells, stem cells and/or stem-like cells and/or cells of the tumor microenvironment such as tumor-associated macrophages (TAMs), immune cells, endothelial cells.

In another aspect, the present disclosure provides for the antibody-drug conjugate or the pharmaceutical composition as described herein, for use in the treatment of a neoplastic disease or disorder.

In some embodiments, the neoplastic disease or disorder is solid tumor or a lymphoma.

In some embodiments, the neoplastic disease or disorder is a cancer.

In some embodiments, the cancer is selected from the group consisting of breast cancer, brain cancer, cancer of the Central Nervous System (CNS), lung cancer, prostate cancer, pancreatic cancer, skin cancer, lymphoma, sarcoma, rhabdoid tumor, cholangiocarcinoma, or a metastasis of any one of said cancer forms. In further embodiments cholangiocarcinoma can be an intrahepatic bile duct cancer, a perihilar bile duct cancer or a distal (extrahepatic) bile duct cancer.

In some embodiments, the breast cancer is selected from the group consisting of triple negative breast cancer and inflammatory breast cancer. Triple negative breast cancer is selected from the group consisting of basal-like 1 breast cancer, basal-like 2 breast cancer, claudin-low breast cancer, metaplastic breast cancer (MBC), interferon-rich breast cancer, immunomodulatory breast cancer, mesenchymal breast cancer, mesenchymal stem-like breast cancer, luminal androgen receptor breast cancer and unstable breast cancer.

In some embodiments, the lung cancer is selected from the group consisting of squamous cell lung carcinoma, lung adenocarcinoma, large cell lung carcinoma and small-cell lung carcinoma.

In some embodiments, the prostate cancer is small cell neuroendocrine carcinoma (SCNC) or castrate-resistant prostate cancer (CRPC).

In some embodiments, the pancreatic cancer is an exocrine tumor or an endocrine tumor, such as wherein the pancreas cancer is an exocrine tumor selected from the group consisting of ductal adenocarcinoma, acinar cell carcinoma, adeno-squamous carcinoma, intraductal papillary mucinous neoplasm (IPMN) and Pancreatic intraepithelial neoplasia. Alternatively, the pancreatic cancer is an endocrine tumor selected from the group consisting of neuroendocrine tumor, gastrinoma, glucaganoma, insulinoma, somatostatinoma, VIPoma, and non-functional Islet cell tumor, such as wherein the neuroendocrine tumor is a grade I, grade II or grade III pancreatic cancer.

In some embodiments, the brain cancer and/or the cancer of the CNS is selected from the group consisting of tumours of neuroepithelial tissue, tumours of cranial and paraspinal nerves, tumours of the meninges, tumours of the haematopoietic system, and tumours of the sellar region.

In some embodiments, the the brain cancer and/or the cancer of the CNS is an astrocytic tumour, such as an astrocytic tumor selected from Glioblastoma, Giant cell glioblastoma, Pilocytic astrocytoma, Pilomyxoid astrocytoma, Subependymal giant cell astrocytoma, Pleomorphic xanthoastrocytoma, Diffuse astrocytoma, Anaplastic astrocytoma, Gliosarcoma, Gliomatosis cerebri.

In some embodiments, the brain cancer and/or the cancer of the CNS is an embryonal tumour such as a neuroblastoma, a medulloblastoma and/or a rhabdoid tumour of the brain.

In some embodiments, the brain cancer and/or the cancer of the CNS is an ependymal tumour, such as an ependymal tumour selected from Subependymoma, Myxopapillary ependymoma, Ependymoma, and Anaplastic ependymoma.

In some embodiments, the tumours of neuroepithelial tissue are selected from:
a) Astrocytic tumours selected from Pilocytic astrocytoma, Pilomyxoid astrocytoma, Subependymal giant cell astrocytoma, Pleomorphic xanthoastrocytoma, Diffuse astrocytoma, Anaplastic astrocytoma, Glioblastoma, Giant cell glioblastoma, Gliosarcoma, Gliomatosis cerebri, and
b) Oligodendroglial tumours selected from Oligodendroglioma and Anaplastic oligodendroglioma, and
c) Oligoastrocytic tumours selected from Oligoastrocytoma and Anaplastic oligoastrocytoma, and
d) Ependymal tumours selected from Subependymoma, Myxopapillary ependymoma, Ependymoma, Anaplastic ependymoma, and
e) Choroid plexus tumours selected from Choroid plexus papilloma, Atypical choroid plexus papilloma, and Choroid plexus carcinoma, and
f) Other neuroepithelial tumours selected from Astroblastoma, Chordoid glioma of the third ventricle, and Angiocentric glioma, and
g) Neuronal and mixed neuronal-glial tumours selected from Dysplastic gangliocytoma of cerebellum (Lhermitte-Duclos), Desmoplastic infantile astrocytoma/ganglioglioma, Dysembryoplastic neuroepithelial tumour, Gangliocytoma, Ganglioglioma, Anaplastic ganglioglioma, Central neurocytoma, Extraventricular neurocytoma, Cerebellar liponeurocytoma, Papillary glioneuronal tumour, Rosette-forming glioneuronal tumour of the fourth ventricle, and Paraganglioma, and
h) Tumours of the pineal region selected from Pineocytoma, Pineal parenchymal tumour of intermediate differentiation, Pineoblastoma, and Papillary tumours of the pineal region, and
i) Embryonal tumours selected from Medulloblastoma, Medulloblastoma with extensive nodularity, Anaplastic medulloblastoma, CNS Primitive neuroectodermal tumour, CNS Neuroblastoma, and Atypical teratoid/rhabdoid tumour.

In some embodiments, the tumours of cranial and paraspinal nerves are selected from:
a) Schwannoma,
b) Neurofibroma,
c) Perineurioma, and
d) Malignant peripheral nerve sheath tumour (MPNST).

In some embodiments, the tumours of the meninges are selected from:
a) Tumours of meningothelial cells, selected from Meningioma, Atypical meningioma, Anaplastic meningioma,
b) Mesenchymal tumours selected from Lipoma, Angiolipoma, Hibernoma, Liposarcoma, Solitary fibrous tumour, Fibrosarcoma, Malignant fibrous histiocytoma, Leiomyoma, Leiomyosarcoma, Rhabdomyoma, Rhabdomyosarcoma, Chondroma, Chondrosarcoma, Osteoma, Osteosarcoma, Osteo-chondroma, Haemangioma, Epithelioid hemangioendothelioma, Haemangiopericytoma, Anaplastic haemangiopericytoma, and Angiosarcoma, Kaposi Sarcoma, Ewing Sarcoma-PNET,
c) Primary melanocytic lesions selected from
d) Diffuse melanocytosis, Melanocytoma, Malignant melanoma, Meningeal melanomatosis, and
e) Other neoplasms related to the meninges such as Haem-angioblastoma.

In some embodiments, the tumours of the haematopoietic system are selected from:
a) Malignant Lymphomas, Plasmocytoma, and
b) Granulocytic sarcoma

In some embodiments, the tumours of the sellar region are selected from:
a) Craniopharyngioma,
b) Granular cell tumour,
c) Pituicytoma, and
d) Spindle cell oncocytoma of the adenohypophysis.

In some embodiments, the skin cancer is a melanoma, such as malignant melanoma.

In some embodiments, the sarcoma is an osteosarcoma.

In some embodiments, the the antibody-drug conjugate as described herein inhibits cell division and/or inhibits cell proliferation and/or inhibits cell survival and/or induces cell death of cells expressing integrin alpha10beta1. The described effect of the antibody-drug conjugate may result from blocking of microtubule polymerization, resulting in cell cycle arrest and inducing caspase-3-dependent apoptosis. Additionally or alternatively, the cell proliferation and/or cell survival may be the result of the antibody-mediated cytotoxicity and/or binding to the epitope antigen of cancer cells and inhibits downstream signal transduction of antigen receptor which can lead to inhibition of cell survival and proliferation and induce cell apoptosis. Additionally or alternatively, the antibody-drug conjugate as described herein may irreversibly bind to DNA and cause strong inter strand cross-linking that prevents DNA strand separation, thus destroying necessary DNA metabolic processes and finally leading to cell deaths.

In some embodiments, the antibody-drug conjugate as described herein inhibits spreading of cancer cells to other sites within the same organ, as well as to other organs. Thus, in some embodiments, the antibody-drug conjugate as described herein inhibits metastasis of cancer, wherein the cancer is any one mentioned herein, such as wherein the cancer is a cancer characterized by expression of integrin alpha10beta1.

In some embodiments, the the antibody-drug conjugate as described herein induces bystander effect which results in cell death of integrin alpha10-negative cancer cells. By “bystander effect” is meant an effect where the cytotoxic agent that is conjugated to the antibody by either a cleavable or non-cleavable linker has the capacity to diffuse across cell membranes after the release from the antibody and thereby cause killing of neighbouring cells. When the cytotoxic agent is conjugated by a cleavable or non-cleavable linker, it may be either the cytotoxic agent only or the cytotoxic agent with a part of the linker that has the bystander kill capacity. The capacity to diffuse across cell membranes is related to the hydrophobicity of the cytotoxic agent or the combination of the cytotoxic agent and the linker. It can also be understood that the term “bystander killing” or “bystander effect” refers to the killing of target negative cells in the presence of target-positive cells, wherein killing of target-negative cells is not observed in the absence of target-positive cells. Cell-to-cell contact, or at least proximity between target-positive and target-negative cells, enables bystander killing. This type of killing is distinguishable from “off-target killing,” which refers to the indiscriminate killing of target-negative cells. “Off-target killing” may be observed in the absence of target-positive cells.

In some embodiments the antibody-drug conjugate or the pharmaceutical composition according to other aspect of present disclosure is administered parenterally, for example, intravenously, intracerebroventricularly, intraarticularly, intra-arterially, intraperitoneally, intrathecally, intraventricularly, intrasternally, intracranially, intramuscularly or subcutaneously, by infusion techniques, or in situ. By administering in situ it can be understood that the antibody-drug conjugate or the pharmaceutical composition may be administered at the site of neoplasm where abnormal cells have not spread beyond where they first formed, or it can be understood that the a the antibody-drug conjugate or the pharmaceutical composition may be administered at the site, from which a neoplasm has been surgically removed.

In some embodiments the antibody-drug conjugate or the pharmaceutical composition is administered in combination with one or more further agents, such as one or more further therapeutic agents.

In one aspect the present disclosure provides for a method for treatment of a disease characterized by expression of integrin alpha10beta1 in a subject, comprising administering to the subject the antibody-drug conjugate or the pharmaceutical composition according to other aspects of the present disclosure described herein.

In one aspect the present disclosure provides for a use of the antibody-drug conjugate or the pharmaceutical composition according other aspects of the disclosure for treatment of a disease.

In one aspect the present disclosure provides for a kit comprising the antibody-drug conjugate or the pharmaceutical composition as described herein, optionally further comprising means for administering the antibody-drug conjugate to a subject and/or instructions for use.

### EXAMPLES

### Example 1. Antibody Production/Humanization

### 1.1 Design of Composite Human Antibody Variable Regions

Aim: To generate human antibody sequence segments to create humanised antibody variants.
Material and Methods: Structural models of the mouse antibody Tm-Ab regions were produced. Based on structure analysis, sequences of humanized variants, likely to be essential for the binding properties of the antibody, were selected and analysed in silico. Human sequence segments were identified both within and outside the CDR regions. Selected sequence segments were assembled to generate complete humanized V region sequences that were devoid of, or reduced in, significant T cell epitopes to avoid immunogenicity.
Results: The design resulted in seven heavy chain (VH1 to VH7) and three light chain (VK1 to VK3) sequences that were used for gene synthesis and expression in mammalian cells (Table 1). Seven VH were combined with 3 VK generating 21 different humanized variants.
Conclusion: Twenty-one humanized variants and one chimeric variant were generated.

TABLE 1
Summary table of the 21 humanized antibody variants
including a chimeric antibody (Th-Ab0).

| | VH0 | VH1 | VH2 | VH3 | VH4 | VH5 | VH6 | VH7 |
|---|---|---|---|---|---|---|---|---|
| VK0 | ✓ | | | | | | | |
| VK1 | | ✓ | ✓ | ✓ | ✓ | ✓ | ✓ | ✓ |
| VK2 | | ✓ | ✓ | ✓ | ✓ | ✓ | ✓ | ✓ |
| VK3 | | ✓ | ✓ | ✓ | ✓ | ✓ | ✓ | ✓ |

1.2 Construction and Transient Expression of Chimeric IgG1 and Humanized lgG1 Variants
Aim: To generate small scale batches of antibodies for lead candidate selection.
Material and Methods: The sequences of chimeric antibody Th-Ab0 and the humanized antibody variants were used to express IgG1 antibodies in CHO cells. Culture supernatants were harvested on day 6 post-transfection and antibody concentrations were measured.
Results and Conclusion: All 21 different variants had higher antibody concentrations compared to the chimeric antibody (Th-Ab0) (data not shown).

### 1.3 Selection of Five Lead Candidates

Aim: To select five lead candidates from the 21 humanized variants based on integrin α10 binding properties.
Material and methods: The mouse myoblast cell line C2C12 overexpressing either human integrin alpha10beta1 (C2C12apha10) or human integrin alpha11beta1 (C2C12alpha11) were used to study antibody binding and specificity. The C2C12 cells were incubated (100 000 cells/sample) with the integrin alpha10 antibodies at 1 μg/ml for 30 min followed by a secondary antibody for 30 min followed by analysis by flow cytometry.
Results: The results showed that the chimeric antibody Th-Ab0 as well as humanized antibody variants Th-Ab1-Th-Ab15 had the best binding affinity to C2C12alpha10 cells (data not shown). None of the antibodies showed binding to the control C2C12alpha11 cells (data not shown). Additionally, risk analysis data from the sequence design process suggested that the antibodies Th-Ab9, Th-Ab11, Th-Ab12, Th-Ab14 and Th-Ab15 have lower immunogenicity than the other variants (data not shown), therefore, these antibodies were selected for further internalization and thermal stability studies.
Conclusion: Five humanized antibody lead candidates with specific and high affinity binding to integrin alpha10beta1, and low immunogenicity risk scores, were selected: Th-Ab9, Th-Ab11, Th-Ab12, Th-Ab14 and Th-Ab15.

### 1.4 Internalization of the Five Antibody Lead Candidates

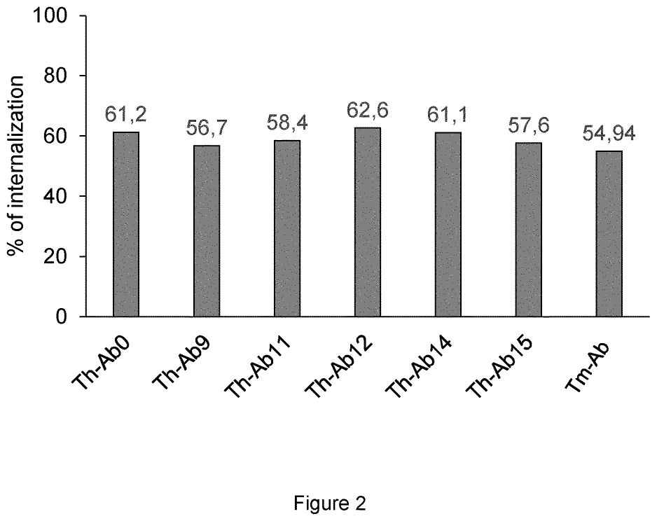

Aim: To investigate the internalization level of the five antibody candidates in integrin alpha10 expressing cell lines.
Materials and Methods: The mouse myoblast cell line C2C12 overexpressing the integrin alpha10beta1 (C2C12alpha10) was used in the internalization assay. The cells (500 000 cells/sample) were incubated with the five lead humanized antibodies as well as the chimeric antibody Th-Ab0 and the original mouse antibody Tm-Ab (1 μg/sample) for 30 min at 4 °C. The cells were then washed with PBS containing 2% FBS and incubated at 37 °C for 90 minutes or 4 hours and then incubated with a secondary antibody for 20 min in the dark at 4 °C. The cells were washed twice with PBS prior to analysis of internalization rate (%) by flow cytometry.
Results: All antibodies were internalized by more than 50% within 4 h of incubation (range of 55-63%) in all the cell lines tested (FIG. 2). Internalization of the humanized antibodies was slightly better than internalization of the mouse antibody Tm-Ab.
Conclusion: The results show that all the five antibody lead candidates were internalized efficiently. This finding supports the therapeutic potential of integrin alpha10-targeting antibody-drug conjugates.

### 1.5 Thermal Stability Analysis

Aim: To rank the top five humanized antibody variants based on stability.
Material and Methods: Thermal stability analysis of the humanized antibody lead candidates was performed using an Uncle™ biostability platform and software. Samples for each variant were formulated in PBS and Sypro Orange at a final concentration of 0.5 mg/ml. Samples were subjected to a thermal ramp from 25-95 °C, with a ramp rate of 0.3 °C/minute and excitation at 473 nm. Monitoring of static light scattering (SLS) at 473 nm allowed the detection of protein aggregation, and calculation of Tagg.
Results: All five humanized antibody lead candidates showed good pharmaceutical properties, including high thermostability and low aggregation propensity which will facilitate manufacturing and storage and suggests long serum half-life. The Tagg appear to be slightly higher for Th-Ab12, Th-Ab11, and Th-Ab9 than the other humanized variants and the chimeric antibody Th-Ab0 (Table 2).
Conclusion: The thermal stability was higher for all the five selected humanized antibody lead candidates compared to the chimeric antibody (Th-Ab0). This further supports the therapeutic potential of the humanized antibodies.

TABLE 2
Summary of thermal stability values for the five lead
variants and chimeric antibody.

| | | | | Variable | Variable |
|---|---|---|---|---|---|
| | AvTₘ₁ | AvTₘ₂ | AvT_{agg} | light | heavy |
| Variant | (°C) | (°C) | (°C) | chain | chain |
| Th-Ab0 | 65.4 | 78.4 | 76.8 | | |
| Th-Ab9 | 64.7 | 79.1 | 77.0 | SEQ ID | SEQ ID |
| | | | | NO: 12 | NO: 15 |
| Th-Ab11 | 64.5 | 80.2 | 78.3 | SEQ ID | SEQ ID |
| | | | | NO: 11 | NO: 16 |
| Th-Ab12 | 64.9 | 81.8 | 79.6 | SEQ ID | SEQ ID |
| | | | | NO: 12 | NO: 16 |
| Th-Ab14 | 66.4 | 78.3 | 76.8 | SEQ ID | SEQ ID |
| | | | | NO: 11 | NO: 17 |
| Th-Ab15 | 66.2 | 78.7 | 76.8 | SEQ ID | SEQ ID |
| | | | | NO: 12 | NO: 17 |

### Example 2. Production and Characterization of Th-Ab12-ADC

Aim: To generate and characterize Th-Ab12-ADC.
Material and Methods: ADCs used for these studies were generated using a well-established conjugation approach. In brief, targeting antibodies were subjected to conjugation to a “anthracycline” type of payload (Glu-(Val-cit-PAB-DMEDA-PNU159682)-6′-amino-β-cyclodextrin) by mild reduction of interchain disulfides, followed by a cysteine rebridging approach to produce ADCs with highly homogeneous drug-to-antibody ratios (DAR) of around 4. ADCs were then purified using a Proteus 5 mL column. Fractions were analysed by LC-MS and SEC and pooled based on average DAR 4±0.3 (LC-MS) and HMWS<10% (SEC). Pooled fractions were buffer exchanged with Dulbecco's PBS, pH 7.1. Concentrated conjugate samples were sterile filtered through a 0.22 μm pore size, PVDF membrane filter. The ADCs were characterised by hydrophobic interaction chromatography (HIC), size-exclusion chromatography (SEC), liquid chromatography-mass spectrometry (LC-MS), SDS-PAGE and quantified by UV. The endotoxin levels were determined by the EndoSafe-PTS platform (Charles River).
Results: ADCs were generated with a disulfide rebridging conjugation technology (bis-mPEG according to Formula C″ and functionalized with a glutamic acid), a cytotoxic payload PNU159682 (Formula X) and a cleavable linker Val-Cit-PAB-DMEDA (Formula Y). The structure is shown in FIG. 1. An average DAR of the ADCs is 4. The percentage of the monomeric purity were over 95% for all the ADCs and the percentage of the free payload species were not detectable. Endotoxin levels were below 0.1 EU/mg.
Conclusion: The ADCs were successfully conjugated with high purity and low endotoxin levels, enabling in vitro and in vivo evaluation.

### Example 3. Binding Affinity and Specificity of Th-Ab12-ADC

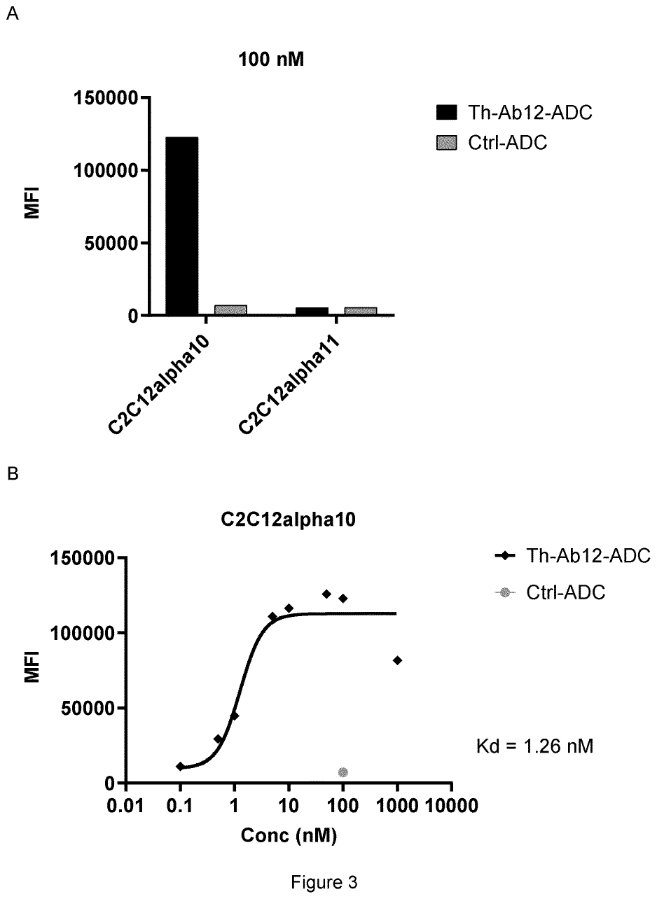

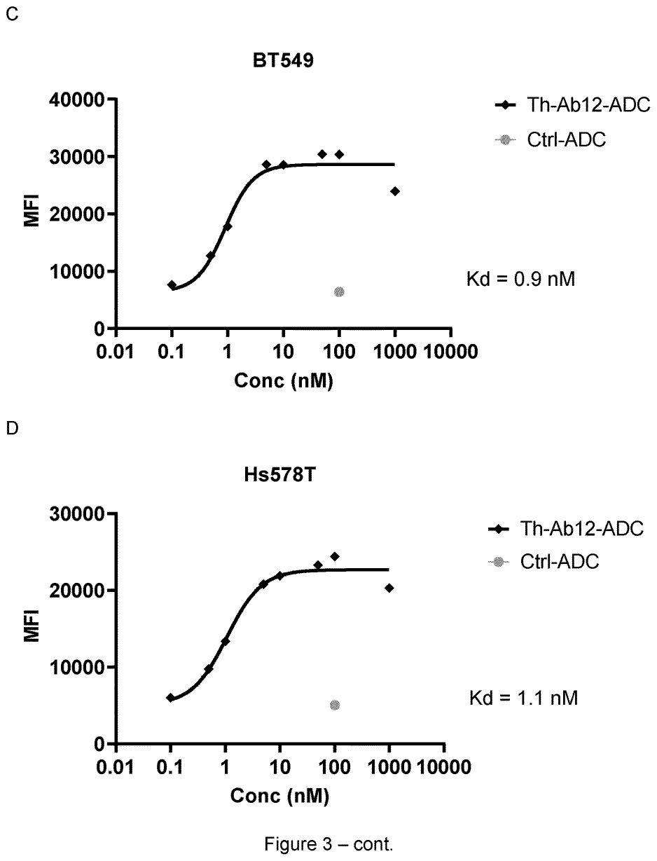

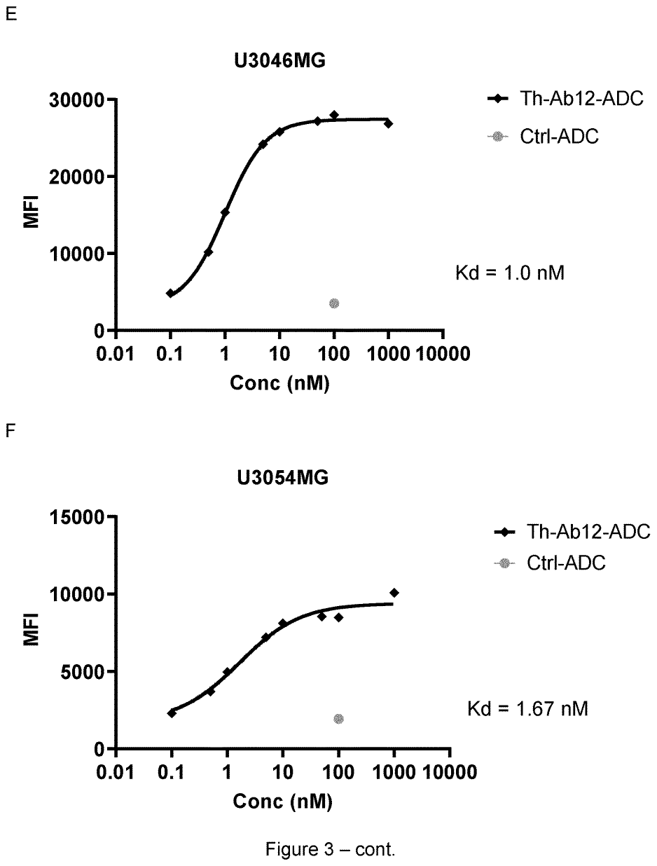

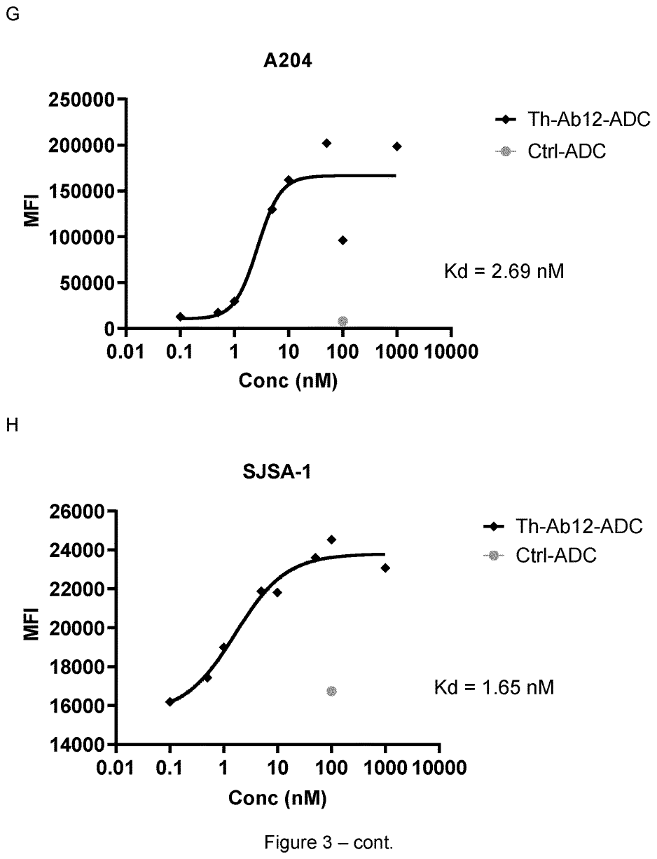

Aim: To investigate the binding affinity and specificity of Th-Ab12-ADC in different cell lines.
Material and Methods: Integrin alpha10beta1 expressing cells (C2C12alpha10), integrin alpha11beta1 overexpressing cells (C2C12alpha11) with no expression of integrin alpha10beta1, triple negative breast cancer cells (BT549 and Hs578T), rhabdoid tumour (A204), osteosarcoma (SJSA-1) and human patient-derived glioblastoma cells (U3046MG and U3054 MG) were used. The cells were incubated with the IgG1 isotype control Ctrl-ADC at 100 nM or Th-Ab12-ADC at different concentrations (0.5, 1, 5, 10, 50, 100, 1000 nM) for 30 min at 4 °C and were then incubated with a secondary antibody for 30 min at 4 °C prior to the analysis by flow cytometry.
Results: The results showed that the Th-Ab12-ADC has high affinity to C2C12alpha10, BT549, Hs578T, U3046MG, U3054MG, A204 and SJSA-1 and binds in a dose dependent manner. The binding EC50 for each cell line is within a range of 0.9-2.69 nM (FIG. 3B-H). There was no binding to C2C12alpha11 cells suggesting that Th-Ab12-ADC is specifically binding to integrin alpha10 (FIG. 3A).
Conclusion: The findings demonstrate that Th-Ab12-ADC binds to integrin alpha10 in a specific manner and with high affinity, which supports its potential for successful drug development.

### Example 4. In Vitro Cytotoxicity of Th-Ab12-ADC

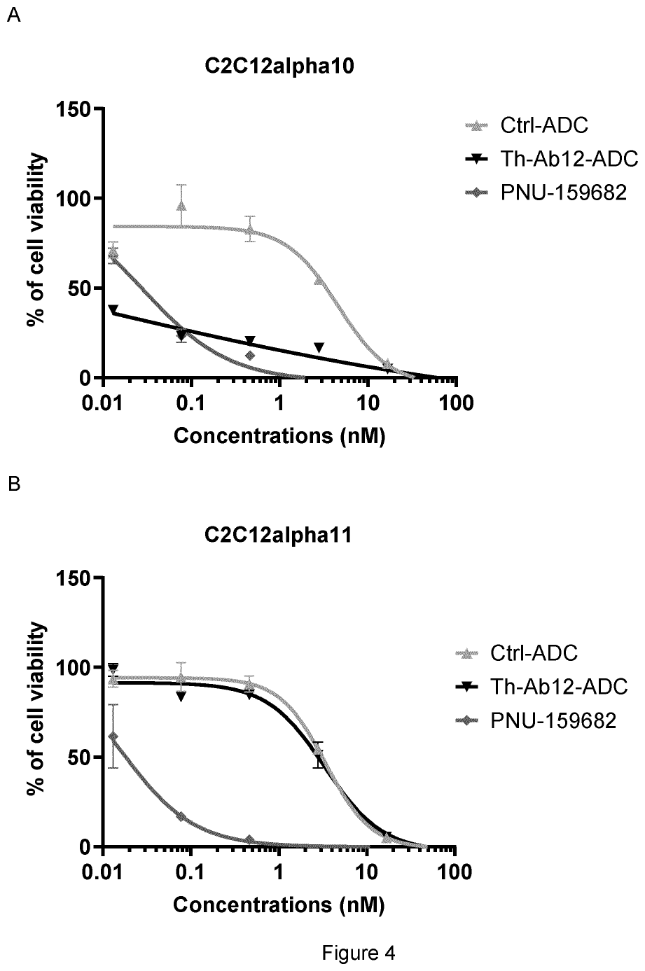

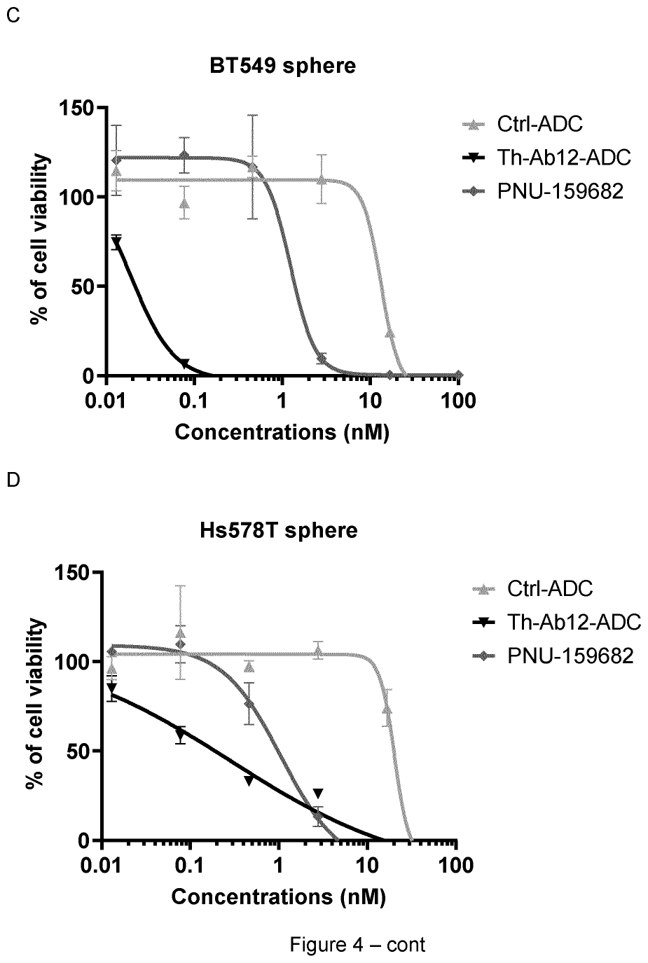

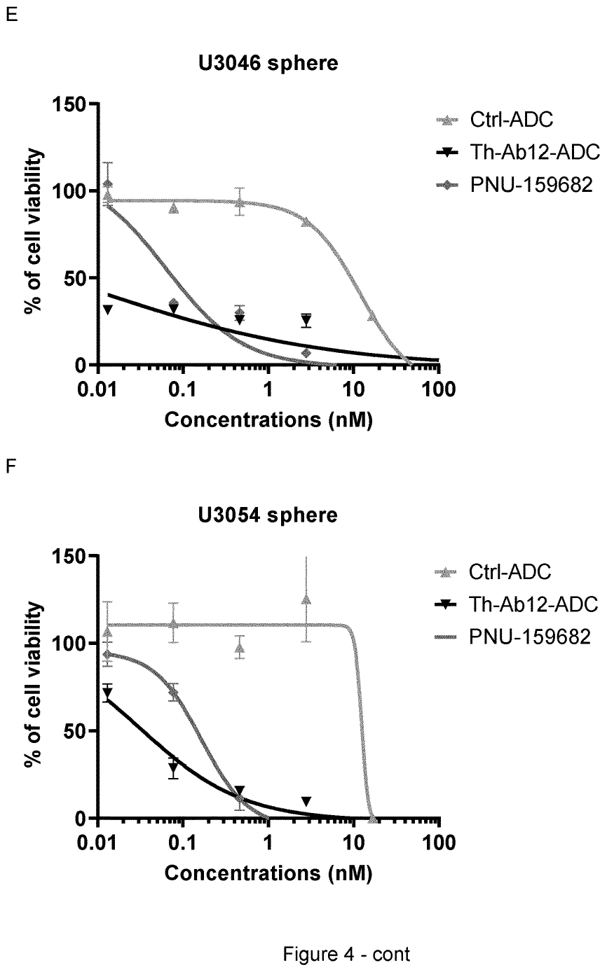

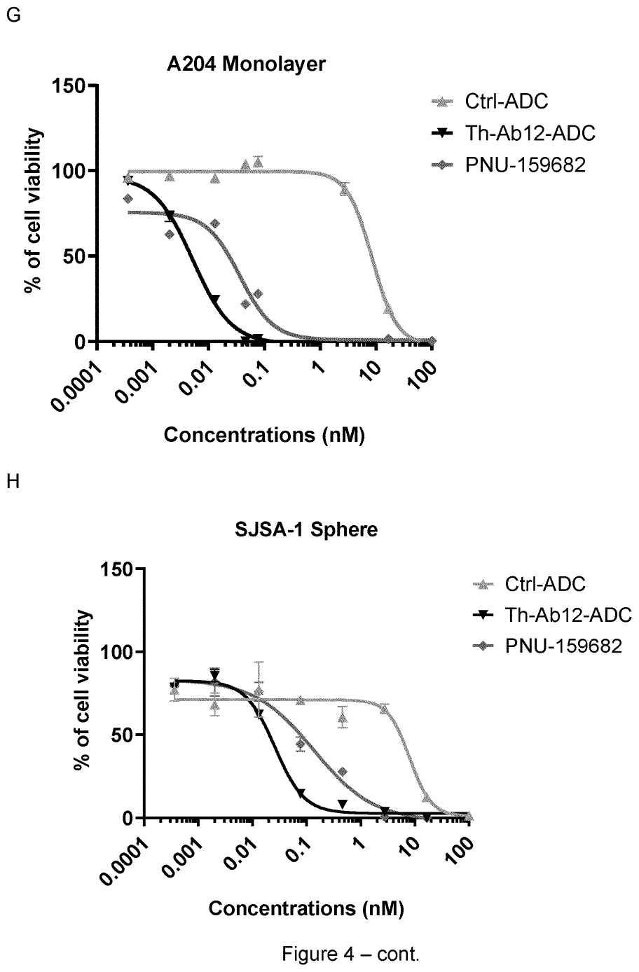

Aim: To evaluate the in vitro efficacy of Th-Ab12-ADC in different cell lines. Material and Methods: Cells were seeded either as monolayer (C2C12alpha10, C2C12alpha11 and A204) or as spheres (BT549, Hs578T, U3046MG, U3054MG and SJSA-1) and treated with the IgG1 isotype control Ctrl-ADC or Th-Ab12-ADC at different concentrations (0.13, 0.77, 4.6, 27.8, 167, 100 nM) for 5 days (C2C12alpha10, C2C12alpha11, A204 and SJSA-1) or 10 days (BT549, Hs578T, U3046MG and U3054MG). On the day of termination, the cells were incubated for 3-4 hours with WST-1, which measures the viable cells, and the plates were read at OD450 nm wavelength via SpectraMax. The dose response curves and IC₅₀ values were generated in GraphPad Prism 9. The WST-1 assay is based on the cleavage of tetrazolium salt WST-1 for formazan by cellular mitochondrial dehydrogenases. The larger the number of viable cells, the higher the activity of the mitochondrial dehydrogenases, and in turn the greater the amount of formazan dye formed.
Results: Th-Ab12-ADC demonstrated toxicity with high potency (IC₅₀=1 μM) in C2C12alpha10 cells while there was no effect in C2C12alpha11 cells, even when tested at therapeutically relevant doses, suggesting an integrin alpha10 specific effect (FIGS. 4A and B). Similar results were observed with the TNBC (BT549 and Hs578T), the GB (U3046MG and U3054MG), rhabdoid tumour A204, and osteosarcoma SJSA-1 cell lines. The free payloads showed less toxicity than the Th-Ab12-ADC in all the tested cell lines (FIG. 3C-H). The differences in IC₅₀ values between control ADC and Th-Ab12-ADC supports a relatively large therapeutic window for each cell line (Table 3).

TABLE 3
To measure the potency of the ADCs and payload, the half-
maximal inhibitory concentration (IC₅₀) for each cell lines
was calculated based on the dose response curves.

| | IC50 (nM) | | |
|---|---|---|---|
| Cell lines | Ctrl-ADC | Th-Ab12-ADC | PNU-159682 |
| C2C12alpha10 | 3.361 | 0.001 | 0.027 |
| C2C12alpha11 | 3.108 | 2.791 | 0.018 |
| BT549 | 13.381 | 0.021 | 1.400 |
| Hs578T | 19.907 | 0.190 | 0.981 |
| U3046MG | 9.410 | 0.003 | 0.077 |
| U3054MG | 12.571 | 0.034 | 0.145 |
| A204 | 8.307 | 0.005 | 0.023 |
| SJSA-1 | 5.309 | 0.021 | 0.077 |

Conclusion: Th-Ab12-ADC was found to induce integrin alpha10 specific toxicity in C2C12alpha10, TNBC and GB cell lines with high potency. The fact that Th-Ab12-ADC induced toxicity at approximately 1000 times lower concentrations than the control ADC indicates the potential for a large therapeutic window.

### Example 5. Internalization of Th-Ab12-ADC

Aim: To study the internalization rate of Th-Ab12-ADC in various integrin alpha10 expressing cell lines.
Material and Methods: C2C12alpha10, BT549, Hs578T, U3046MG and U3054MG cells were used. The cells (500 000 cells/sample) were incubated with Th-Ab12-ADC or unconjugated antibody Th-Ab12 (1 μg/0.1 ml) for 30 min on ice. The cells were then washed with PBS containing 2% FBS and incubated at 37 °C for 90 minutes or 4 hours and then incubated with a secondary antibody containing Alexa 488 fluorochrome for 20 min at 4 °C. The cells were washed twice with PBS prior to the analysis of internalization rate (%) by flow cytometry.
Results: Th-Ab12-ADC was internalized by 50-60% within 4 h of incubation in all the cell lines tested (FIG. 4). Overall, the internalization level was higher at 4 h than at 90 min. The internalization of Th-Ab12-ADC was slightly better than unconjugated Th-Ab12 in TNBC and GB cell lines (FIG. 4).
Conclusion: The results show that both the unconjugated antibody Th-Ab12 and Th-Ab12-ADC are internalized to a high degree within 4 h. Th-Ab12-ADC internalized better than the unconjugated antibody Th-Ab12 in TNBC and GB cells.

### Example 6. Th-Ab12-ADC Induces Cell Death/Apoptosis

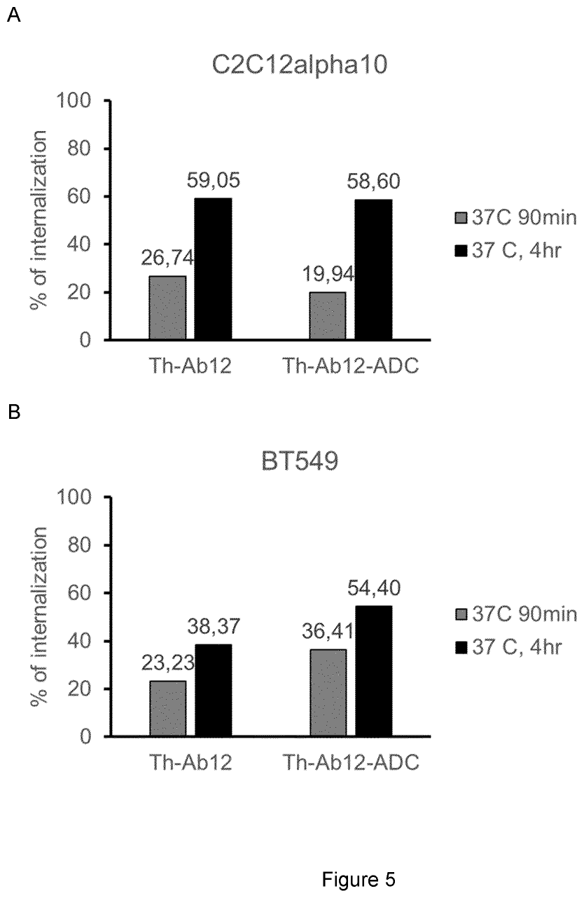

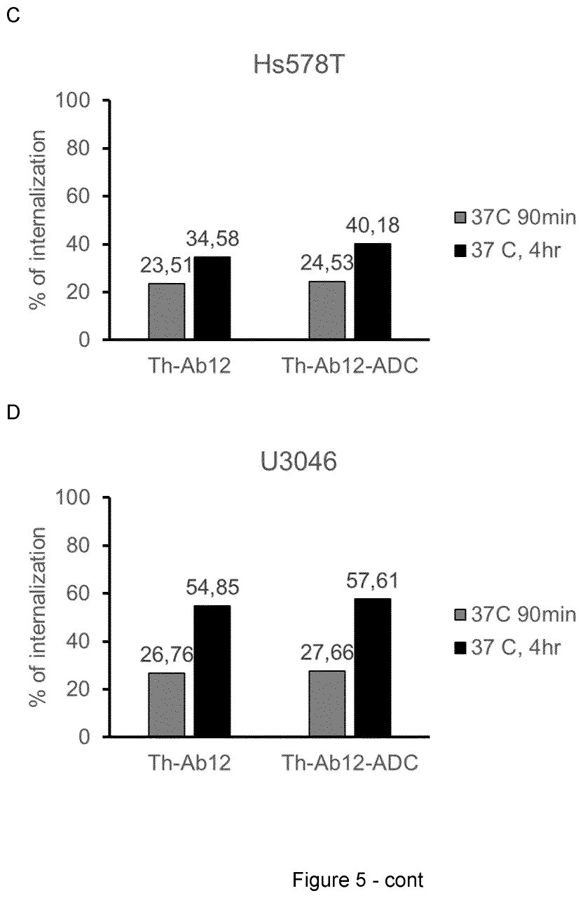

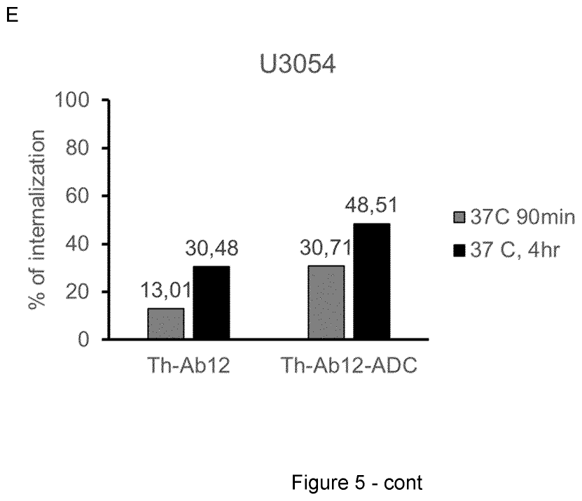

Aim: To investigate the effect of Th-Ab12-ADC on cell cycle distribution and cell death/apoptosis in C2C12alpha10 cells.
Material and Methods: C2C12alpha10 cells (9 000 cells/well) were seeded and cultured as monolayer in a 6-well culture plate and incubated with ADCs at concentration of 0.02 nM. On day 5, the cells were harvested and stained with propidium iodide (PI) before analysis of cell cycle distribution using Propidium Iodide Flow Cytometry Kit (Abcam, ab139418) according to the manufacturer's instructions.
Results: Th-Ab12-ADC treatment led to significant cell death (shown by the percentage of Sub-G1) compared to non-treated cells (NT) and control ADC (Ctrl-ADC) treatment (FIG. 5).
Conclusion: The results suggest that Th-Ab12-ADCs bind to the C2C12alpha10 cells, is internalized, and subsequently cleaved to release the cell toxin. The released toxin then causes DNA damage and lead to cell death/apoptosis.

### Example 7. Bystander Effect of Th-Ab12-ADC

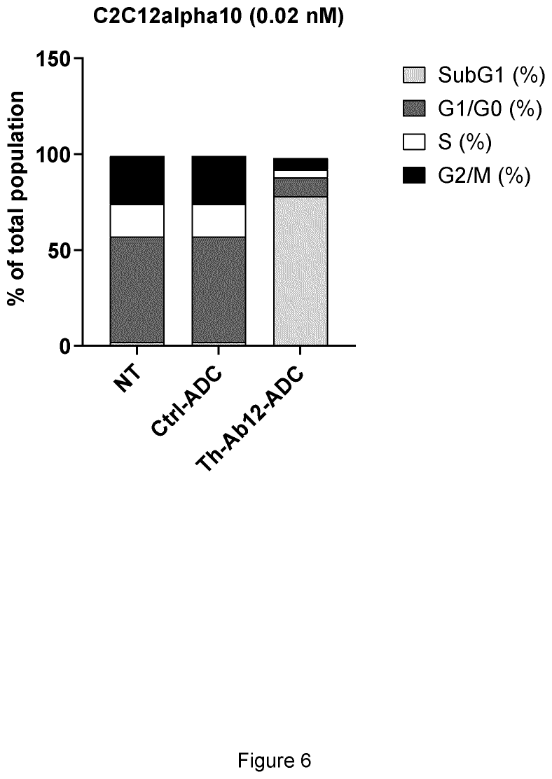

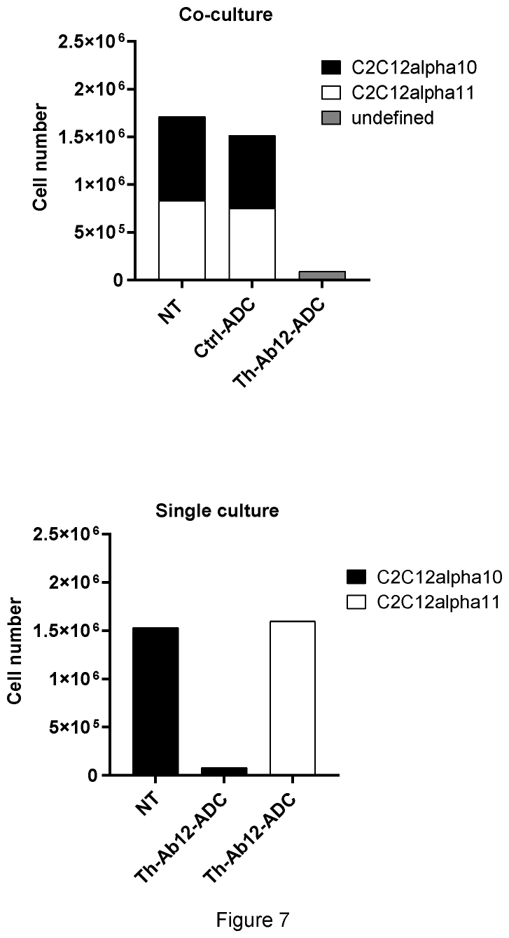

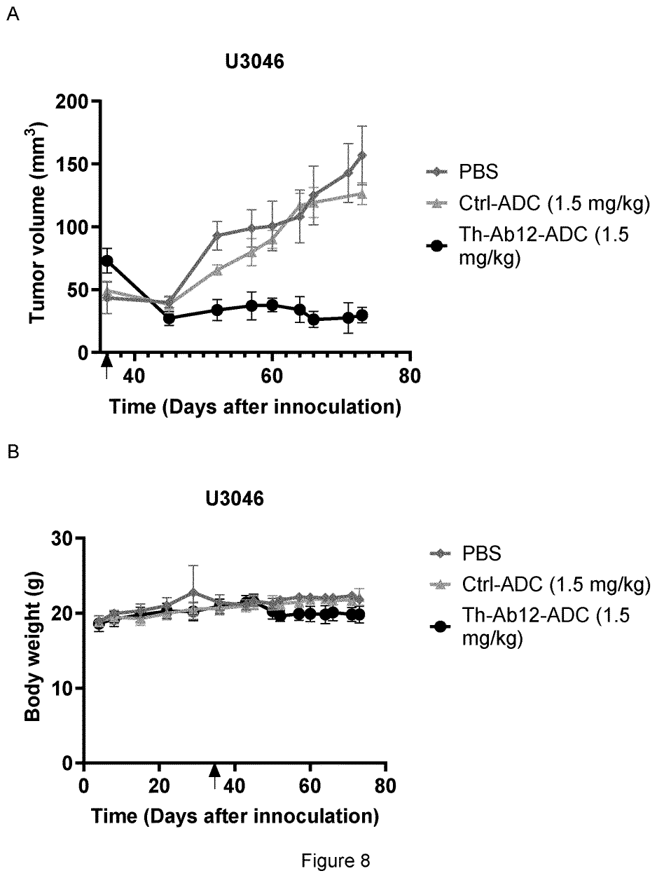

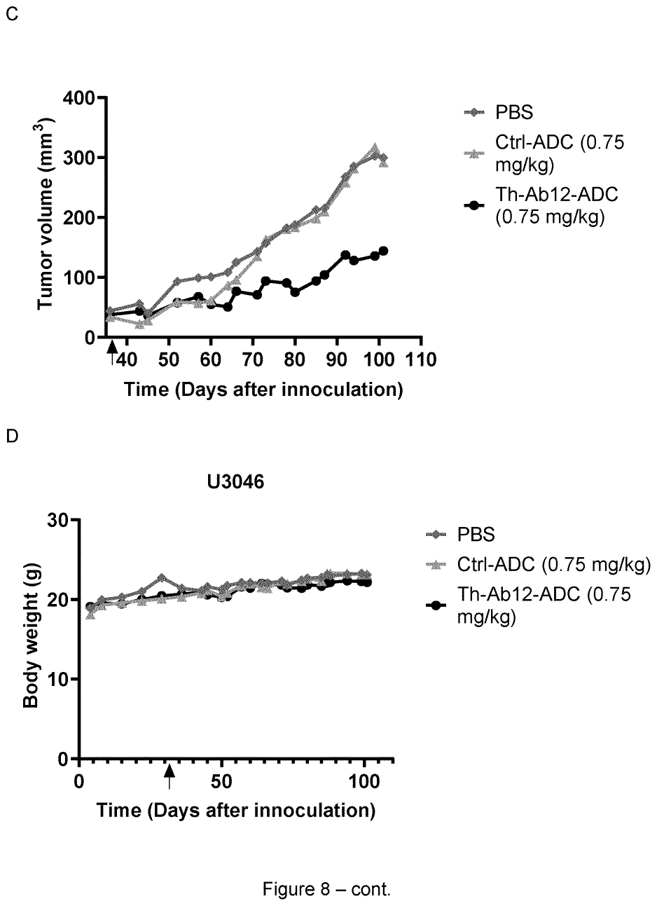

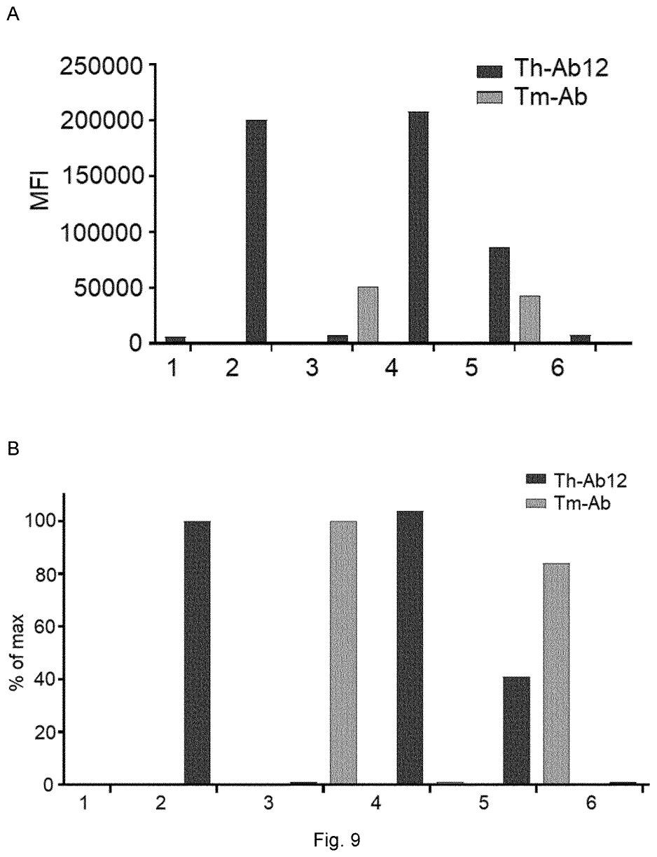

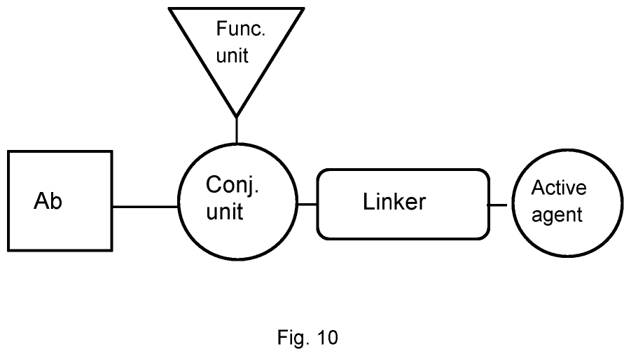

Aim: To investigate if cells not expressing integrin alpha10 will be affected by Th-Ab12-ADC treatment when co-cultured with integrin alpha10 expressing cells, indicating a bystander effect of Th-Ab12-ADC.
Material and Methods: C2C12alpha10 and C2C12alpha11 cells (with no integrin alpha10 expression) were used. C2C12alpha10 and C2C12alpha11 cells were seeded and cultured in a 6-well culture plate at 12 000 and 8 000 cells/well, respectively. In the wells containing only one cell line (C2C12alpha10 or C2C12alpha11), the cells were seeded at 20 000 cells/well. Th-Ab12-ADC and Ctrl-ADC were added to the wells at a concentration of 0.02 nM. Viable cells were detached from the plate after 5 days of culture, and the cell number in each well was determined using a cell counter. In order to determine the ratio of C2C12alpha10 and C2C12alpha11 cells, the cells (100 000/sample) were stained with integrin alpha10 antibody conjugated with Alexa 647 and analyzed by a flow cytometry.
Results: The results demonstrated that Th-Ab12-ADC induces cytotoxicity in both integrin alpha10 positive cells and in integrin alpha10 negative cells when co-cultured (FIG. 6A). There was no toxicity in integrin alpha10 negative cells (C2C12alpha11) when incubated with Th-Ab12-ADC in the absence of integrin alpha10 positive cells (FIG. 6B).
Conclusion: The results demonstrate that Th-Ab12-ADC has good bystander effect which means that Th-Ab12-ADC can also induce cell death in nearby cells without integrin alpha10 expression. This can be beneficial for the treatment of solid tumours with heterogeneous integrin alpha10 expression.

### Example 8. In Vivo Efficacy of Th-Ab12-ADC

Aim: To evaluate the in vivo efficacy of Th-Ab12-ADC in glioblastoma cell line U3046MG-derived xenograft nude mouse model.
Material and Methods: U3046MG cells (1.5×10⁶ cell) containing 30% Matrigel (total volume 100 μl) were injected subcutaneously into the right dorsal flank of four weeks old NMRI-nu immunodeficient mice (Janvier, France). The tumour volume (using a caliper) and body weights were measurement twice a week. Five weeks after inoculation when the mean tumour volume was about 50 mm³, the mice were randomly allocated to different treatment groups based on the tumour volume. The mice were then treated with a single intravenous injection of Th-Ab12-ADC (0.75 mg/kg or 1.5 mg/kg), Ctrl-ADC (0.75 mg/kg or 1.5 mg/kg) or PBS and tumour volumes and body weights were measured biweekly.

### Results:

Treatment with Th-Ab12-ADC inhibited tumour growth compared to the Ctrl-ADC and PBS (FIGS. 8A and C). The inhibitory effect was seen with both doses (0.75 mg/kg and 1.5 mg/kg). The ADC treatments did not affect the body weights of the mice compared to the PBS group (FIGS. 8B and D).
Conclusion: The results show that Th-Ab12-ADC exhibits good in vivo efficacy in glioblastoma U3046MG xenograft nude mouse model. This suggests that Th-Ab 12-ADC has the potential to become an effective treatment for glioblastoma and other aggressive cancers expressing integrin alpha10beta1.

### Example 9. Bind Affinity of Tm-Ab Versus Th-Ab12

### Materials and Methods

Transduced cell lines: The mouse myoblast cell line C2C12 transduced with integrin α10 vector (C2C12a10) was cultured in Dulbecco's Modified Eagle Medium (Gibco) supplemented with 10% FBS (Gibco) and Antibiotic-Antimycotic (100 U/mL, Gibco). The C2C12a10 cells were selected using G418 (1 mg/ml, Gibco).
Antibodies: Primary antibodies: Th-Ab12 (supernatant VH4VK3 variant) and Tm-Ab-A647 (conjugated to AlexaFluor A647, thus Tm-Ab-A647; also corresponding to: “an alternative mouse monoclonal antibody against integrin alpha10” used in Examples 2, 3, 4, and 9 of WO 2020/212416, and to the antibody against integrin alpha10 used in Munksgaard Thorèn M., 2019 [8] both for FACS and conjugated to Saporin) at 10 μg/ml. Secondary antibody: Donkey anti human IgG Alexa 488 (T011), 1:1000 for Th-Ab12.
Flow cytometry: In this first experiment only C2C12a10 cells were used.
Immunostaining of the cells (100 000 cells/sample) was performed by incubating cells with the different antibodies in incubation periods of 30 min at 4 degrees. Samples included are listed in table 3.

TABLE 3

| | 1^{st} | 2^{nd} | 3^{rd} |
|---|---|---|---|
| Sample name | incubation | incubation | incubation |
| 1: unstained | — | — | — |
| 2: Th-Ab12 | Th-Ab12 | Anti-hu IgG | — |
| | | AF488 | |
| 3: Tm-Ab-A647 | Tm-Ab-A647 | — | — |
| 4: (1st. Th-Ab12 2nd. | Th-Ab12 | Anti-hu IgG | Tm-Ab-A647 |
| Tm-Ab-A647) | | AF488 | |
| 5: (1st. Tm-Ab-A647 | Tm-Ab-A647 | Th-Ab12 | Anti-hu IgG |
| 2nd. Th-Ab12) | | | AF488 |
| 6: (2^{nd} Ab only) | — | Anti-hu IgG | — |
| | | AF488 | |

Cells were incubated with a primary antibody against integrin α10 (Th-Ab12 or Tm-Ab-A647) at a concentration of 10 μg/ml. After 30 min incubation with primary antibody, cells were washed twice with FACS buffer; DPBS (Hyclone) containing 1% FBS (Gibco) and 0.1% sodium azide (G Bioscience). Secondary antibody or donkey anti-human IgG AF488 was then added according to the table above and incubated for 30 min. After incubation cells were washed twice with FACS buffer. Finally, a third antibody was added to samples 4 and 5 for the last incubation according to the table above. Samples 4 and 5 were also washed twice prior to the analysis using flow cytometry.

### Results

The samples with only one integrin α10 antibody (samples 2 and 3) are used as single stain reference to see how the primary antibody binds to the target in absence of competition. The samples with successive addition of the two antibodies against integrin α10 (sample 4 and 5) are the samples that will be competing for binding of target in this assay. Samples 1 and 6 are used to determine background staining.

The results in FIGS. 9A and B show that in C2C12a10 cells, when Th-Ab12 antibody was incubated first (Sample 4), Th-Ab12 showed the same binding degree as the single staining (Sample 2) while Tm-Ab did not show any binding. When Tm-Ab antibody was incubated first and then incubated with Th-Ab12 antibody (Sample 5), the binding signal for Tm-Ab decreased with 20% and Th-Ab12 bound to 40% (FIG. 9B). This indicates that Th-Ab12 competed out a certain amount of Tm-Ab and has a higher affinity towards C2C12a10 cells than Tm-Ab.

### CONCLUSION

Based on the results of this study, Th-Ab12 has a higher affinity towards C2C12a10 cells than Tm-Ab.

Sequence overview
SEQ ID NO: 1: RSSQSLVHSNGNTYLH
Variable light chain complementarity-determining
region 1 (CDR-L1)
SEQ ID NO: 2: KVSNRFS
Variable light chain complementarity-determining
region 2 (CDR-L2)
SEQ ID NO: 3: SQSTHVPYT
Variable light chain complementarity-determining
region 3 (CDR-L3)
SEQ ID NO: 4: EYYII
Variable heavy chain complementarity-determining
region 1 (CDR-H1)
SEQ ID NO: 5: EYYVI
Variable heavy chain complementarity-determining
region 1 (CDR-H1)
SEQ ID NO: 6: EYSII
Variable heavy chain complementarity-determining
region 1 (CDR-H1)
SEQ ID NO: 7: EYGII
Variable heavy chain complementarity-determining
region 1 (CDR-H1)
SEQ ID NO: 8: WIFPGSGRTYYSEKFRG
Variable heavy chain complementarity-determining
region 2 (CDR-H2)
SEQ ID NO: 9: DNYGSSGKFFAY
Variable heavy chain complementarity-determining
region 3 (CDR-H3)
Variable light chain - VK1
SEQ ID NO: 10
DVVMTQIPLSLPVTLGQPASISCRSSQSLVHSNGNTYLHWYQQKPGQPPK
LLIYKVSNRFSGVPDRFSGSGSGTDFTLKISRVEAEDVGVYFCSQSTHVP
YTFGQGTKLEIK
Variable light chain - VK2
SEQ ID NO: 11
DVVMTQSPLSLPVTLGQPASISCRSSQSLVHSNGNTYLHWYQQKPGQPPK
LLIYKVSNRFSGVPDRFSGSGSGTDFTLKISRVEAEDVGVYFCSQSTHVP
YTFGQGTKLEIK
Variable light chain - VK3
SEQ ID NO: 12
DVVMTQSPLSLPVTLGQPASISCRSSQSLVHSNGNTYLHWYQQKPGQPPK
LLIYKVSNRFSGVPDRFSGSGSGTDFTLKISRVEAEDVGVYYCSQSTHVP
YTFGQGTKLEIK
Variable heavy chain - VH1
SEQ ID NO: 13
QVQLVQSGPELKKPGASVKISCKTSGYTFTEYYIIWVKQRPGQGLEWLGW
IFPGSGRTYYSEKFRGRATLTVDKSTSTAYMLLSSLTSEDSAVYFCARDN
YGSSGKFFAYWGQGTLVTVSS
Variable heavy chain - VH2
SEQ ID NO: 14
QVQLVQSGAEVKKPGASVKISCKTSGYTFTEYYIIWVKQRPGQGLEWLGW
IFPGSGRTYYSEKFRGRATLTVDKSTSTAYMELSSLRSEDTAVYFCARDN
YGSSGKFFAYWGQGTLVTVSS
Variable heavy chain - VH3
SEQ ID NO: 15
QVQLVQSGAEVKKPGASVKVSCKTSGYTFTEYYIIWVRQAPGQGLEWLGW
IFPGSGRTYYSEKFRGRATITVDKSTSTAYMELSSLRSEDTAVYYCARDN
YGSSGKFFAYWGQGTLVTVSS
Variable heavy chain - VH4
SEQ ID NO: 16
QVQLVQSGAEVKKPGASVKVSCKTSGYTFTEYYIIWVRQAPGQGLEWLGW
IFPGSGRTYYSEKFRGRVTITADKSTSTAYMELSSLRSEDTAVYYCARDN
YGSSGKFFAYWGQGTLVTVSS
Variable heavy chain - VH5
SEQ ID NO: 17
QVQLVQSGAEVKKPGASVKVSCKTSGYTFTEYYVIWVRQAPGQGLEWLGW
IFPGSGRTYYSEKFRGRATITVDKSTSTAYMELSSLRSEDTAVYYCARDN
YGSSGKFFAYWGQGTLVTVSS
Variable heavy chain - VH6
SEQ ID NO: 18
QVQLVQSGAEVKKPGASVKVSCKTSGYTFTEYSIIWVRQAPGQGLEWLGW
IFPGSGRTYYSEKFRGRATITVDKSTSTAYMELSSLRSEDTAVYYCARDN
YGSSGKFFAYWGQGTLVTVSS
Variable heavy chain - VH7
SEQ ID NO: 19
QVQLVQSGAEVKKPGASVKVSCKTSGYTFTEYGIIWVRQAPGQGLEWLGW
IFPGSGRTYYSEKFRGRATITVDKSTSTAYMELSSLRSEDTAVYYCARDN
YGSSGKFFAYWGQGTLVTVSS
Constant light chain
SEQ ID NO: 20
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC
Constant heavy chain
SEQ ID NO: 21
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK

### REFERENCES

1. Dean, A. Q.; Luo, S.; Twomey, J. D.; Zhang, B. Targeting cancer with antibody-drug conjugates: Promises and challenges. MAbs 2021, 13, doi: 10.1080/19420862.2021.1951427.
2. Giugliano, F.; Corti, C.; Tarantino, P.; Michelini, F.; Curigliano, G. Bystander effect of antibody-drug conjugates: fact or fiction? Curr. Oncol. Rep. 2022, doi: 10.1007/s11912-022-01266-4.
3. Gullberg, D. E.; Lundgren-Åkerlund, E. Cottagen-binding I domain integrins-What do they do? Prog. Histochem. Cytochem. 2002, 37, 3-54, doi: 10.1016/S0079-6336 (02) 80008-0.
4. Camper, L.; Holmvall, K.; Wängnerud, C.; Aszódi, A.; Lundgren-Åkerlund, E. Distribution of the collagen-binding integrin α1031 during mouse development. Cell Tissue Res. 2001, 306, 107-116, doi: 10.1007/s004410100385.
5. Varas, L.; Ohlsson, L. B.; Honeth, G.; Olsson, A.; Bengtsson, T.; Wiberg, C.; Bockermann, R.; Järnum, S.; Richter, J.; Pennington, D.; et al. α 10 Integrin Expression Is Up-Regulated on Fibroblast Growth Factor-2-Treated

Mesenchymal Stem Cells with Improved Chondrogenic Differentiation Potential. Stem Cells Dev. 2007, 16, 965-978, doi: 10.1089/scd.2007.0049.
6 Lundgren-Åkerlund, E.; Aszòdi, A. Integrin α10ß1: a collagen receptor critical in skeletal development. In Advances in experimental medicine and biology, 2014; Vol. 819, pp. 61-71 ISBN 9789401791526.
7. Uvebrant, K.; Reimer Rasmusson, L.; Talts, J.; Alberton, P.; Aszodi, A.; Lundgren-Åkerlund, E. Integrin α10ß1-selected Equine MSCs have Improved Chondrogenic Differentiation, Immunomodulatory and Cartilage Adhesion Capacity. Ann Stem Cell Res. 2019, 2, 001-009.
8. Thorén, M. M.; Masoumi, K. C.; Krona, C.; Huang, X.; Kundu, S.; Schmidt, L.; Forsberg-nilsson, K.; Keep, M. F.; Englund, E.; Nelander, S.; et al. Integrin α 10, a Novel Therapeutic Target in Glioblastoma, Regulates Cell Migration, Proliferation, and Survival. Cancers (Basel). 2019, 11, 587.
9. Masoumi, K. C.; Huang, X.; Sime, W.; Mirkov, A.; Munksgaard, M.; Massoumi, R.; Lundgren-Åkerlund, E. Integrin α 10-Antibodies Reduce Glioblastoma Tumor Growth and Cell Migration. Cancers (Basel). 2021, 13, 1184, doi: https://www.mdpi.com/2072-6694/13/5/1184.
10. Bumbaca, D.; Boswell, C. A.; Fielder, P. J.; Khawli, L. A. Physiochemical and biochemical factors influencing the pharmacokinetics of antibody therapeutics. AAPS J. 2012, 14, 554-558, doi: 10.1208/s12248-012-9369-y.

1. An antibody or antigen-binding fragment thereof with binding specificity for integrin alpha10, wherein the antibody or antigen-binding fragment comprises:
a light chain variable region comprising
a) a CDR-L1 comprising or consisting of an amino acid sequence of SEQ ID NO: 1;
b) a CDR-L2 comprising or consisting of an amino acid sequence of SEQ ID NO: 2; and
c) a CDR-L3 comprising or consisting of an amino acid sequence of SEQ ID NO: 3;
and
a heavy chain variable region comprising
d) a CDR-H1 comprising or consisting of an amino acid sequence selected from the group consisting of SEQ ID NO: 4, SEQ ID NO:5, SEQ ID NO:6 and SEQ ID NO:7;
e) a CDR-H2 comprising or consisting of an amino acid sequence of SEQ ID NO: 8; and
f) a CDR-H3 comprising or consisting of an amino acid sequence of SEQ ID NO: 9.

2. The antibody or antigen-binding fragment thereof according to claim 1, wherein the antibody or antigen-binding fragment comprises:
a) an immunoglobulin light chain variable region comprising or consisting of the amino acid sequence selected from the group consisting of SEQ ID NO: 10, SEQ ID NO: 11 and SEQ ID NO: 12 or a sequence having at least 85% sequence identity to any of SEQ ID NO: 10, SEQ ID NO: 11 and SEQ ID NO: 12, such as at least 95%, such as 98% or 99% sequence identity to any of SEQ ID NO: 10, SEQ ID NO: 11 and SEQ ID NO: 12; and/or
b) an immunoglobulin heavy chain variable region comprising or consisting of the amino acid sequence selected from the group consisting of SEQ ID NO: 13, SEQ ID NO: 14, SEQ ID NO: 15, SEQ ID NO: 16, SEQ ID NO: 17, SEQ ID NO: 18 and SEQ ID NO: 19 or a sequence having at least 85% sequence identity to any of SEQ ID NO: 13, SEQ ID NO: 14, SEQ ID NO: 15, SEQ ID NO: 16, SEQ ID NO: 17, SEQ ID NO: 18 and SEQ ID NO: 19, such as at least 95%, such as 98% or 99% sequence identity to any of SEQ ID NO: 13, SEQ ID NO: 14, SEQ ID NO: 15, SEQ ID NO: 16, SEQ ID NO: 17, SEQ ID NO: 18 and SEQ ID NO: 19.

3. The antibody or antigen-binding fragment thereof according to any one of the preceding claims, wherein the antibody or antigen-binding fragment comprises:
a) an immunoglobulin light chain variable region comprising or consisting of the amino acid sequence of SEQ ID NO: 12 and an immunoglobulin heavy chain variable region comprising or consisting of the amino acid sequence of SEQ ID NO: 16; or
b) an immunoglobulin light chain variable region comprising or consisting of the amino acid sequence of SEQ ID NO: 12 and an immunoglobulin heavy chain variable region comprising or consisting of the amino acid sequence of SEQ ID NO: 15; or
c) an immunoglobulin light chain variable region comprising or consisting of the amino acid sequence of SEQ ID NO: 11 and an immunoglobulin heavy chain variable region comprising or consisting of the amino acid sequence of SEQ ID NO: 16; or
d) an immunoglobulin light chain variable region comprising or consisting of the amino acid sequence of SEQ ID NO: 11 and an immunoglobulin heavy chain variable region comprising or consisting of the amino acid sequence of SEQ ID NO: 17; or
e) an immunoglobulin light chain variable region comprising or consisting of the amino acid sequence of SEQ ID NO: 12 and an immunoglobulin heavy chain variable region comprising or consisting of the amino acid sequence of SEQ ID NO: 17.

4. The antibody or antigen-binding fragment thereof according to any one of the preceding claims, wherein the antibody or antigen-binding fragment comprises:
a) an immunoglobulin light chain variable region comprising CDR-L1 of SEQ ID NO: 1, CDR-L2 of SEQ ID NO:2 and CDR-L3 of SEQ ID NO:3; and
b) an immunoglobulin heavy chain variable region comprising
i. CDR-H1 of SEQ ID NO:4, CDR-H2 of SEQ ID NO:8 and CDR-H3 of SEQ ID NO:9, or
ii. CDR-H1 of SEQ ID NO:5, CDR-H2 of SEQ ID NO:8 and CDR-H3 of SEQ ID NO:9.

5. The antibody or antigen-binding fragment thereof according to any one of the preceding claims, wherein the antibody or antigen-binding fragment comprises:
a) an immunoglobulin light chain variable region comprising or consisting of the amino acid sequence of SEQ ID NO: 12; and
b) an immunoglobulin heavy chain variable region comprising or consisting of the amino acid sequence of SEQ ID NO: 16;

6. The antibody or antigen-binding fragment thereof according to any one of the preceding claims, wherein the antibody or antigen-binding fragment comprises: a light chain variable region comprising
a) a CDR-L1 comprising or consisting of an amino acid sequence of SEQ ID NO: 1;
b) a CDR-L2 comprising or consisting of an amino acid sequence of SEQ ID NO: 2; and
c) a CDR-L3 comprising or consisting of an amino acid sequence of SEQ ID NO: 3;
and a heavy chain variable region comprising
d) a CDR-H1 comprising or consisting of an amino acid sequence of SEQ ID NO: 4;
e) a CDR-H2 comprising or consisting of an amino acid sequence of SEQ ID NO: 8; and
f) a CDR-H3 comprising or consisting of an amino acid sequence of SEQ ID NO: 9.

7. The antibody or antigen-binding fragment thereof according to any one of the preceding claims, wherein the integrin alpha10 polypeptide is a part of an integrin alpha10beta1 heterodimer.

8. The antibody or antigen-binding fragment thereof according to any one of the preceding claims, wherein the integrin alpha10beta1 is human integrin alpha10beta1.

9. The antibody or antigen-binding fragment thereof according to any one of the preceding claims, wherein the integrin alpha10beta1 is expressed on the surface of a cell.

10. The antibody or antigen-binding fragment thereof according to any one of the preceding claims, wherein the antibody or antigen-binding fragment thereof binds to the extracellular I-domain of the integrin alpha10 subunit.

11. The antibody or antigen-binding fragment thereof according to any one of the preceding claims, wherein the antibody is selected from a mouse antibody, a chimeric antibody, a human antibody, a humanised antibody, a humanised antigen-binding fragment, a Fab fragment, a Fab′ fragment, an F(ab′)2 fragment, an Fv, a single chain antibody (SCA) such as an scFv, a disulphide-bonded Fv, the variable portion of the heavy and/or light chains thereof, and a Fab miniantibody.

12. The antibody or antigen-binding fragment thereof according to any one of the preceding claims, wherein the antibody is a monoclonal antibody or antigen-binding fragment thereof.

13. The antibody or antigen-binding fragment thereof according to any one of the preceding claims, wherein the antibody is a humanised or fully human monoclonal antibody or antigen-binding fragment thereof.

14. The antibody or antigen-binding fragment thereof according to any one of the preceding claims, wherein the antibody is a recombinant antibody or antigen-binding fragment thereof.

15. The antibody or antigen-binding fragment thereof according to any one of the preceding claims, wherein the antibody or antigen-binding fragment thereof is conjugated to an additional moiety.

16. The antibody or antigen-binding fragment thereof according to claim 15, wherein the additional moiety comprises a detectable moiety, such as a detectable moiety selected from the group consisting of a fluorophore, an enzyme and a radioactive tracer or radioisotope.

17. A polynucleotide encoding an antibody or antigen-binding fragment thereof according to any one of the preceding claims or a component polypeptide chain thereof.

18. The polynucleotide according to claim 17, wherein the molecule is a cDNA molecule.

19. The polynucleotide according to any one of claims 17 to 18, encoding an antibody light chain or variable region thereof.

20. The polynucleotide according to any one of claims 17 to 18 encoding an antibody heavy chain or variable region thereof.

21. The polynucleotide according to any one of claims 17 to 20 encoding an antibody according to any one of claims 1 to 16.

22. A vector comprising a polynucleotide according to any one of the claims 17 to 21.

23. A recombinant host cell comprising a polynucleotide according to any one of claims 17 to 21 or a vector according to claim 22.

24. A method for producing an antibody or antigen-binding fragment thereof according to any one of claims 1 to 16, the method comprising culturing a host cell according to claim 23 comprising the polynucleotide according to any one of the claims 17 to 21 or the vector according to claim 22, under conditions which permit expression of the encoded antibody or antigen-binding fragment thereof.

25. An in vitro method for the detection of cells expressing integrin alpha10beta1 in a subject, the method comprising:
a) providing a sample of cells from a subject to be tested, such as biopsy tissue or blood sample;
b) optionally, extracting and/or purifying the cells present in the sample;
c) contacting an antibody or antigen-binding fragment thereof according to any one of items 1 to 16 with cells present in the sample;
d) determining whether the antibody or antigen-binding fragment thereof binds to the cells
wherein the binding of the antibody or antigen-binding fragment thereof to the cells is indicative of the presence of a disease or disorder associated with cells expression integrin alpha10 in the tissue of a subject.

26. An in vitro method for identifying a patient with a disease or disorder associated with cells expressing integrin alpha10 who would benefit from treatment with an antibody or antigen-binding fragment thereof according to any one of claims 1 to 16, the method comprising:
a) providing a sample, such as biopsy tissue or blood sample from a patient to be tested;
b) optionally, extracting and/or purifying the cells present in the sample;
c) contacting an antibody or antigen-binding fragment thereof according to any one of claims 1 to 16 with the sample;
d) determining whether the antibody or antigen-binding fragment thereof binds to an integrin alpha10 subunit in the sample,
wherein the binding of the antibody or antigen-binding fragment thereof to an integrin alpha10 subunit is indicative of a patient who would benefit from treatment with an antibody or antigen-binding fragment thereof according to any one of claims 1 to 16.

27. A method for the detection of cells expressing integrin alpha10, the method comprising:
c) contacting an antibody or antigen-binding fragment thereof according to any one of claims 1 to 16 with cells to be analysed for their expression of integrin alpha10;
d) determining whether the antibody or antigen-binding fragment thereof binds to the cells
wherein the binding of the antibody or antigen-binding fragment thereof to the cells is indicative of the presence of a disease or disorder associated with cells expression integrin alpha10 in the tissue of a subject.

28. The method according to claim 27, wherein the method is an in vivo method or an in vitro method.

29. A method for in vivo imaging the expression of the integrin alpha10beta1 in a mammal, the method comprising the steps of
a) Providing a mammal,
b) Providing an antibody or antigen-binding fragment thereof according to any one of claims 1 to 16,
c) administering the antibody or antigen-binding fragment thereof according to any one of claims 1 to 16 to the mammal so as to allow the antibody or a fragment thereof to bind to an extracellular domain of integrin alpha10beta1 of cells in said mammal,
d) optionally adding a second labelled antibody or a fragment thereof to the sample, wherein the second antibody or a fragment thereof binds to the antibody or a fragment thereof in c),
e) detecting the antibody or antigen-binding fragment thereof according to any one of claims 1 to 16 of said cells in c), or optionally detecting the second labelled antibody or a fragment thereof in d) bound to the antibody or a fragment thereof, and
f) creating an image of the detected antibody or a fragment thereof, thereby imaging the expression of integrin alpha10beta1 on cells in a mammal in vivo.

30. An antibody-drug conjugate directed against integrin alpha10 comprising:
a) an antibody or antigen-binding fragment thereof according to any one of the preceding claims 1 to 16,
b) an active agent, and
c) optionally, a linker which links a) to b).

31. The antibody-drug conjugate according to claim 30, wherein the active agent is a therapeutic agent, a cytotoxic agent, a microtubule toxin or a transcription toxin.

32. The antibody-drug conjugate according to claim 30, wherein the active agent is a cytotoxic agent.

33. The antibody-drug conjugate according to claim 31, wherein
the active agent is a therapeutic agent selected from the group consisting of microtubule toxins, immunomodulators, nucleotide-based agents, DNA toxins and transcription toxins.

34. The antibody-drug conjugate according to claim 30, wherein the active agent is a microtubule toxin, such as a microtubule toxin selected from the group consisting of Auristatin-based toxins, Maytansinoid-based toxins, Tubulysins-based toxins and Eribulin.

35. The antibody-drug conjugate according to claim 30, wherein the active agent is a transcription toxin, such as an RNA polymerase II and/or Ill inhibiting agent.

36. The antibody-drug conjugate according to any one of claims 30 to 35, wherein the active agent is a therapeutic agent selected from the group consisting of alkylating agents, anthracyclines, antimetabolites, anti-microtubule/anti-mitotic agents, histone deacetylase inhibitors, kinase inhibitors, peptide antibiotics, platinum-based antineoplastics, topoisomerase inhibitors and cytotoxic antibiotics.

37. The antibody-drug conjugate according to claim 30, wherein the active agent is a transcription toxin selected from the group consisting of Doxorubicin, Doxorubicin derivatives and Amanitin.

38. The antibody-drug conjugate according to any one of claims 30 and/or 37, wherein the active agent is an anthracycline, such as an anthracycline selected from Daunorubicin, doxorubicin, epirubicin, idarubicin, and 3′-deamino-3″-4′-anhydro-[2″(S)-methoxy-3″(R)-hydroxy-4″-morpholinyl] doxorubicin (PNU159682).

39. The antibody-drug conjugate according to any one of claims 30 and 38, wherein the active agent is 3′-deamino-3″-4′-anhydro-[2″(S)-methoxy-3″(R)-hydroxy-4″-morpholinyl] doxorubicin (PNU159682).

40. The antibody-drug conjugate according to any one of claims 30 to 39, having a drug-to-antibody ratio (DAR) between 1 and 10, such as between 2 and 8, for example between 3 and 6, such as 1, 2, 3 or 4.

41. The antibody-drug conjugate according to any one of claims 30 to 40, wherein the linker is an enzyme-cleavable linker.

42. The antibody-drug conjugate according to any one of claims 30 to 41, wherein the linker is an enzyme-cleavable linker, such as a cathepsin cleavable linker.

43. The antibody-drug conjugate according to any one of claims 30 to 42, wherein the linker comprises: one or more self-immolative molecules, such as p-aminobenzylcarbamoyl (PAB) and N,N′-Dimethylethylenediamine (DMEDA).

44. The antibody-drug conjugate according to any one of claims 30 to 43, wherein the linker comprises a dipeptide, such as Valine-Citrulline (Val-Cit) or Valine-Alanine (Val-Ala).

45. The antibody-drug conjugate according to any one of claims 30 to 44, wherein the linker comprises a dipeptide and one or more self-immolative molecules, such as p-aminobenzylcarbamoyl (PAB).

46. The antibody-drug conjugate according to any one of claims 30 to 45, wherein the linker comprises a dipeptide and one or more self-immolative molecules, such as p-aminobenzylcarbamoyl (PAB) N,N′-Dimethylethylenediamine (DMEDA).

47. The antibody-drug conjugate according to any one of claims 30 to 46, wherein the linker comprises a dipeptide, such as Valine-Citrulline (Val-Cit) or Valine-Alanine (Val-Ala), and the self-immolative molecules p-aminobenzylcarbamoyl (PAB) and N,N′-Dimethylethylenediamine (DMEDA).

48. The antibody-drug conjugate according to any one of claims 30 to 47, wherein wherein the linker comprises or consists of Val-Cit-PAB-DMEDA.

49. The antibody-drug conjugate according to any one of claims 30 to 48, wherein the antibody-drug conjugate comprises a conjugating unit, such as a conjugating unit derived from a reactive group such as a functionalized benzoic acid, an activated carboxylic acid derivative, an amino group, a maleimide group or derivatives thereof, N-hydroxysuccinimide, bis-sulfones, azides and alkynes.

50. The antibody-drug conjugate according to any one of claims 30 to 49,
wherein the conjugating unit is derived from a functionalized bis-sulfone group, such as a bis-sulfone group according to Formula C 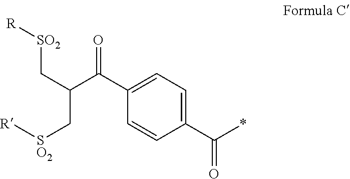
wherein * indicates an attachment site to a linker, and wherein R and R′ are each independently selected form the group consisting of selected from the group consisting of C₁-C₆ alkyl, a C₃-C₇ cycloalkyl, a C₃-C₇ heterocycloalkyl, phenyl, C₅-C₁₀ aryl, each of which may optionally be substituted by one or more selected from halogen, cyano, amino, C₁-C₆ alkyl, C₁-C₆ alkoxy, phenyl, and C5-C10 aryl.

51. The antibody-drug conjugate according to any one of claims 30 to 49,
wherein the conjugating unit is derived from a functionalized bis-sulfone group according to Formula C″ (bis-mPEG): 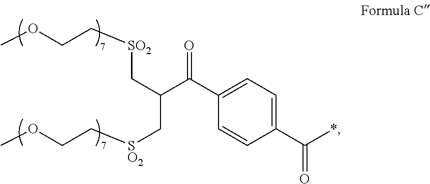
wherein * indicates an attachment site to a linker.

52. The antibody-drug conjugate according to any one of claims 30 to 51, wherein the conjugating unit is derived from a functionalized bis-sulfone group of Formula C″ (bis-mPEG) and wherein said conjugating unit is functionalized with a glutamic acid (bis-mPEG-Glu).

53. The antibody-drug conjugate according to any one of claims 30 to 52, wherein the antibody-drug conjugate comprises a functional unit, such as a unit that improves solubility of the antibody-drug conjugate, such as a cyclodextrin or a PEG molecule.

54. The antibody-drug conjugate according to any one of claims 30 to 53, wherein the functional unit comprises or consists of 6′-amino-β-cyclodextrin.

55. The antibody-drug conjugate according to any one of claims 30 to 54, wherein the functional unit comprises or consists of a PEG molecule having a molecular weight of 10 kDa or below.

56. The antibody-drug conjugate according to any one of claims 30 to 55, wherein the functional unit, such as the unit that improves solubility of the antibody-drug conjugate comprises or consists of a PEG molecule consisting of 72 PEG units or less.

57. The antibody-drug conjugate according to any one of claims 30 to 56, wherein the functional unit is attached to the conjugating unit.

58. The antibody-drug conjugate according to any one of claims 30 to 57, wherein the functional unit is attached to the glutamic acid comprised in the conjugating unit.

59. The antibody-drug conjugate according to any one of claims 30 to 58, wherein the functional unit is attached to the linker.

60. The antibody-drug conjugate according to any one of claims 30 to 59, wherein the antibody-drug conjugate comprises:
bis-mPEG-Glu-(Val-cit-PAB-DMEDA-PNU159682)-6′-amino-β-cyclodextrin, as in Formula A 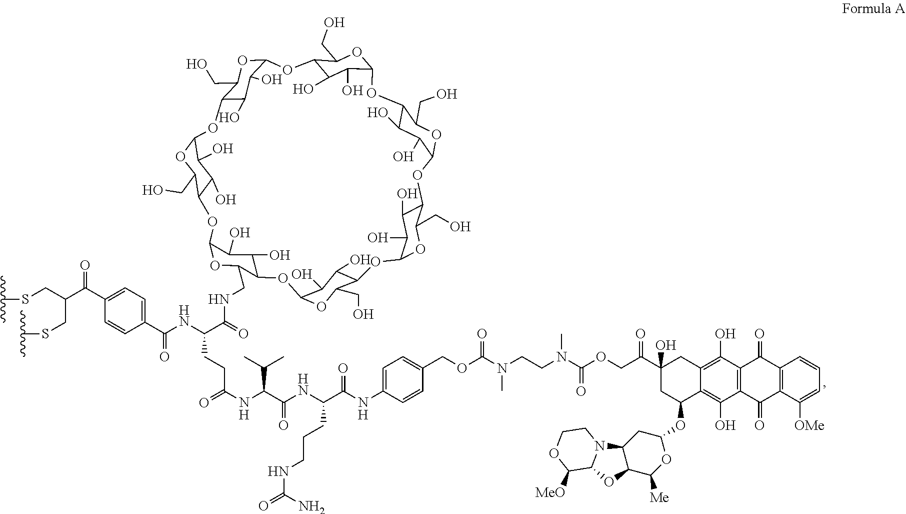 [SMILES: *SCC(CS*)C(=O)c1ccc(C(=O)N[C@@H](CCC(=O)N[C@H](C(=O)N[C@@H](CCCNC(N)=O)C(=O)Nc2ccc(COC(=O)N(C)CCN(C)C(=O)OCC(=O)[C@]3(O)Cc4c(O)c5c(c(O)c4[C@@H](O[C@H]4C[C@H]6[C@H](O[C@@H]7[C@@H](OC)OCCN76)[C@H](C)O4)C3)C(=O)c3c(OC)cccc3C5=O)cc2)C(C)C)C(=O)NCC2OC3OC4C(CO)OC(OC5C(CO)OC(OC6C(CO)OC(OC7C(CO)OC(OC8C(CO)OC(OC9C(CO)OC(OC2C(O)C3O)C(O)C9O)C(O)C8O)C(O)C7O)C(O)C6O)C(O)C5O)C(O)C4O)cc1]
wherein [symbol] indicates the attachment to an antibody or antigen-binding fragment thereof.

61. The antibody-drug conjugate according to any one of claims 30 to 60, wherein the antibody or antigen-binding fragment thereof comprises: a light chain variable region comprising
i. a CDR-L1 comprising or consisting of an amino acid sequence of SEQ ID NO: 1;
ii. a CDR-L2 comprising or consisting of an amino acid sequence of SEQ ID NO: 2; and
iii. a CDR-L3 comprising or consisting of an amino acid sequence of SEQ ID NO: 3;
and a heavy chain variable region comprising
iv. a CDR-H1 comprising or consisting of an amino acid sequence of SEQ ID NO: 4;
v. a CDR-H2 comprising or consisting of an amino acid sequence of SEQ ID NO: 8; and
vi. a CDR-H3 comprising or consisting of an amino acid sequence of SEQ ID NO: 9.

62. The antibody-drug conjugate according to any one of claims 30 to 61, wherein the antibody or antigen-binding fragment comprises:
i. an immunoglobulin light chain variable region comprising or consisting of the amino acid sequence of SEQ ID NO: 12; and
ii. an immunoglobulin heavy chain variable region comprising or consisting of the amino acid sequence of SEQ ID NO: 16.

63. The antibody-drug conjugate according to any one of claims 30 to 62, wherein the antibody-drug conjugate comprises:
a) the antibody or antigen-binding fragment thereof according to claim 1, wherein the antibody or antigen-binding fragment thereof comprises
a light chain variable region comprising a CDR-L1 consisting of SEQ ID NO: 1, a CDR-L2 consisting of SEQ ID NO:2 and a CDR-L3 consisting of SEQ ID NO:3; and
a heavy chain variable region comprising a CDR-H1 consisting of any one of SEQ ID NO: 4, SEQ ID NO:5, SEQ ID NO:6 and SEQ ID NO:7, a CDR-H2 consisting of SEQ ID NO:8 and a CDR-H3 consisting of SEQ ID NO: 9; and
b) a conjugating unit, such as a conjugating unit derived from a bis-sulfone group according to formula C″ and functionalized with a glutamic acid (bis-mPEG-Glu);
c) a linker comprising or consisting of Val-cit-PAB-DMEDA;
d) a functional unit, such as 6′-amino-β-cyclodextrin; and
e) the active agent PNU159682.

64. A pharmaceutical composition comprising: the antibody or antigen-binding fragment thereof according to any one of claims 1 to 16, or the antibody-drug conjugate according to any one claims 30 to 63, and a pharmaceutically acceptable buffer, diluent, carrier, adjuvant or excipient.

65. A method for delivery of an active agent to a cell expressing alpha10beta1 comprising administering to said cell the antibody-drug conjugate according to any one of claims 30 to 63, or the pharmaceutical composition according to claim 64, such that the active agent is delivered to said cell.

66. The antibody-drug conjugate according to any one claims 30 to 63, or the pharmaceutical composition according to claim 64 for use as a medicament.

67. The antibody-drug conjugate according to any one claims 30 to 63, or the pharmaceutical composition according to claim 64 for use in treating a patient with a disease or disorder associated with cells expressing integrin alpha10.

68. The antibody-drug conjugate according to any one claims 30 to 63, or the pharmaceutical composition according to claim 64 wherein the cells expressing integrin alpha10 are malignant cells or tumor-associated cells, such as cancer associated fibroblast (CAFs), stromal cells, stem cells and/or stem-like cells and/or cells of the tumor microenvironment such as tumor-associated macrophages (TAMs), immune cells, endothelial cells.

69. The antibody-drug conjugate according to any one claims 30 to 63, or the pharmaceutical composition according to claim 64 for use in the treatment of a neoplastic disease or disorder.

70. The antibody-drug conjugate or the pharmaceutical composition according to claim 69, wherein the neoplastic disease or disorder is solid tumor, a lymphoma or a cancer.

71. The antibody-drug conjugate or the pharmaceutical composition according to claim 70, wherein the cancer is selected from the group consisting of breast cancer, brain cancer, cancer of the Central Nervous System (CNS), lung cancer, prostate cancer, pancreatic cancer, skin cancer, lymphoma, sarcoma, rhabdoid tumor, cholangiocarcinoma, or a metastasis of any one of said cancer forms.

72. The antibody-drug conjugate or the pharmaceutical composition according to claim 71, wherein the breast cancer is selected from the group consisting of triple negative breast cancer and inflammatory breast cancer.

73. The antibody-drug conjugate or the pharmaceutical composition according to claim 72, wherein the triple negative breast cancer is selected from the group consisting of basal-like 1 breast cancer, basal-like 2 breast cancer, claudin-low breast cancer, metaplastic breast cancer (MBC), interferon-rich breast cancer, immunomodulatory breast cancer, mesenchymal breast cancer, mesenchymal stem-like breast cancer, luminal androgen receptor breast cancer and unstable breast cancer.

74. The antibody-drug conjugate or the pharmaceutical composition according to claim 71, wherein the lung cancer is selected from the group consisting of squamous cell lung carcinoma, lung adenocarcinoma, large cell lung carcinoma and small-cell lung carcinoma.

75. The antibody-drug conjugate or the pharmaceutical composition according to claim 71, wherein the prostate cancer is small cell neuroendocrine carcinoma (SCNC) or castrate-resistant prostate cancer (CRPC).

76. The antibody-drug conjugate or the pharmaceutical composition according to claim 71, wherein the pancreatic cancer is an exocrine tumor selected from the group consisting of ductal adenocarcinoma, acinar cell carcinoma, adeno-squamous carcinoma, intraductal papillary mucinous neoplasm (IPMN) and Pancreatic intraepithelial neoplasia.

77. The antibody-drug conjugate or the pharmaceutical composition according to claim 71, wherein the pancreatic cancer is an endocrine tumor selected from the group consisting of neuroendocrine tumor, gastrinoma, glucaganoma, insulinoma, somatostatinoma, VIPoma, and non-functional Islet cell tumor, such as wherein the neuroendocrine tumor is a grade I, grade II or grade III pancreatic cancer.

78. The antibody-drug conjugate or the pharmaceutical composition according to claim 71, wherein the brain cancer and/or the cancer of the CNS is selected from the group consisting of tumours of neuroepithelial tissue, tumours of cranial and paraspinal nerves, tumours of the meninges, tumours of the haematopoietic system, and tumours of the sellar region.

79. The antibody-drug conjugate or the pharmaceutical composition according to claim 71, wherein the brain cancer and/or the cancer of the CNS is an astrocytic tumour, such as an astrocytic tumor selected from Glioblastoma, Giant cell glioblastoma, Pilocytic astrocytoma, Pilomyxoid astrocytoma, Subependymal giant cell astrocytoma, Pleomorphic xanthoastrocytoma, Diffuse astrocytoma, Anaplastic astrocytoma, Gliosarcoma, Gliomatosis cerebri.

80. The antibody-drug conjugate or the pharmaceutical composition according to claim 71, wherein the brain cancer and/or the cancer of the CNS is an embryonal tumour such as a neuroblastoma, a medulloblastoma and/or a rhabdoid tumour of the brain.

81. The antibody-drug conjugate or the pharmaceutical composition according to claim 71, wherein the brain cancer and/or the cancer of the CNS is an ependymal tumour selected from Subependymoma, Myxopapillary ependymoma, Ependymoma, and Anaplastic ependymoma.

82. The antibody-drug conjugate or the pharmaceutical composition according to claim 71, wherein the skin cancer is a melanoma, such as malignant melanoma.

83. The antibody-drug conjugate or the pharmaceutical composition according to claim 71, wherein the sarcoma is an osteosarcoma.

84. The antibody-drug conjugate or the pharmaceutical composition according to any one of claims 30 to 83, wherein the antibody-drug conjugate inhibits cell division and/or inhibits cell proliferation and/or inhibits cell survival and/or induces cell death of cells expressing integrin alpha10beta1.

85. The antibody-drug conjugate or the pharmaceutical composition according to any one of claims 30 to 84, wherein the antibody-drug conjugate inhibits spreading of cancer cells to other sites within the same organ, to other sites in a different organ, and/or metastasis of a cancer.

86. The antibody-drug conjugate or the pharmaceutical composition according to any one of claims 30 to 85, wherein the antibody-drug conjugate induces bystander effect which results in cell death of integrin alpha10-negative cancer cells.

87. The antibody-drug conjugate or the pharmaceutical composition according to any one of the preceding claims, wherein the antibody-drug conjugate or the pharmaceutical composition is administered parenterally, for example, intravenously, intracerebroventricularly, intraarticularly, intra-arterially, intraperitoneally, intrathecally, intraventricularly, intrasternally, intracranially, intramuscularly or subcutaneously, by infusion techniques, or in situ.

88. The antibody-drug conjugate or the pharmaceutical composition according to any one of the preceding claims, wherein the antibody-drug conjugate or the pharmaceutical composition is administered in combination with one or more further agents, such as one or more further therapeutic agents.

89. A method for treatment of a disease characterized by expression of integrin alpha10beta1 in a subject, comprising administering to the subject the antibody-drug conjugate according to any one of claims 30 to 63, or the pharmaceutical composition according to claim 64.

90. Use of the antibody-drug conjugate according to any one of claims 30 to 63, or the pharmaceutical composition according to claim 64 for treatment of a disease.

91. A kit comprising the antibody-drug conjugate according to any of claims 30 to 63, or the pharmaceutical composition according to claim 64 optionally further comprising means for administering the antibody-drug conjugate to a subject and/or instructions for use.


SEQUENCE LISTING (21 sequences)

SEQ ID NO: 1 - protein, 16 aa, synthetic construct
RSSQSLVHSNGNTYLH

SEQ ID NO: 2 - protein, 7 aa, synthetic construct
KVSNRFS

SEQ ID NO: 3 - protein, 9 aa, synthetic construct
SQSTHVPYT

SEQ ID NO: 4 - protein, 5 aa, synthetic construct
EYYII

SEQ ID NO: 5 - protein, 5 aa, synthetic construct
EYYVI

SEQ ID NO: 6 - protein, 5 aa, synthetic construct
EYSII

SEQ ID NO: 7 - protein, 5 aa, synthetic construct
EYGII

SEQ ID NO: 8 - protein, 17 aa, synthetic construct
WIFPGSGRTYYSEKFRG

SEQ ID NO: 9 - protein, 12 aa, synthetic construct
DNYGSSGKFFAY

SEQ ID NO: 10 - protein, 112 aa, synthetic construct
DVVMTQIPLSLPVTLGQPASISCRSSQSLVHSNGNTYLHWYQQKPGQPPKLLIYKVSNRFSGVPDRFSGSGSGTDFTLKISRVEAEDVGVYFCSQSTHVPYTFGQGTKLEIK

SEQ ID NO: 11 - protein, 112 aa, synthetic construct
DVVMTQSPLSLPVTLGQPASISCRSSQSLVHSNGNTYLHWYQQKPGQPPKLLIYKVSNRFSGVPDRFSGSGSGTDFTLKISRVEAEDVGVYFCSQSTHVPYTFGQGTKLEIK

SEQ ID NO: 12 - protein, 112 aa, synthetic construct
DVVMTQSPLSLPVTLGQPASISCRSSQSLVHSNGNTYLHWYQQKPGQPPKLLIYKVSNRFSGVPDRFSGSGSGTDFTLKISRVEAEDVGVYYCSQSTHVPYTFGQGTKLEIK

SEQ ID NO: 13 - protein, 121 aa, synthetic construct
QVQLVQSGPELKKPGASVKISCKTSGYTFTEYYIIWVKQRPGQGLEWLGWIFPGSGRTYYSEKFRGRATLTVDKSTSTAYMLLSSLTSEDSAVYFCARDNYGSSGKFFAYWGQGTLVTVSS

SEQ ID NO: 14 - protein, 121 aa, synthetic construct
QVQLVQSGAEVKKPGASVKISCKTSGYTFTEYYIIWVKQRPGQGLEWLGWIFPGSGRTYYSEKFRGRATLTVDKSTSTAYMELSSLRSEDTAVYFCARDNYGSSGKFFAYWGQGTLVTVSS

SEQ ID NO: 15 - protein, 121 aa, synthetic construct
QVQLVQSGAEVKKPGASVKVSCKTSGYTFTEYYIIWVRQAPGQGLEWLGWIFPGSGRTYYSEKFRGRATITVDKSTSTAYMELSSLRSEDTAVYYCARDNYGSSGKFFAYWGQGTLVTVSS

SEQ ID NO: 16 - protein, 121 aa, synthetic construct
QVQLVQSGAEVKKPGASVKVSCKTSGYTFTEYYIIWVRQAPGQGLEWLGWIFPGSGRTYYSEKFRGRVTITADKSTSTAYMELSSLRSEDTAVYYCARDNYGSSGKFFAYWGQGTLVTVSS

SEQ ID NO: 17 - protein, 121 aa, synthetic construct
QVQLVQSGAEVKKPGASVKVSCKTSGYTFTEYYVIWVRQAPGQGLEWLGWIFPGSGRTYYSEKFRGRATITVDKSTSTAYMELSSLRSEDTAVYYCARDNYGSSGKFFAYWGQGTLVTVSS

SEQ ID NO: 18 - protein, 121 aa, synthetic construct
QVQLVQSGAEVKKPGASVKVSCKTSGYTFTEYSIIWVRQAPGQGLEWLGWIFPGSGRTYYSEKFRGRATITVDKSTSTAYMELSSLRSEDTAVYYCARDNYGSSGKFFAYWGQGTLVTVSS

SEQ ID NO: 19 - protein, 121 aa, synthetic construct
QVQLVQSGAEVKKPGASVKVSCKTSGYTFTEYGIIWVRQAPGQGLEWLGWIFPGSGRTYYSEKFRGRATITVDKSTSTAYMELSSLRSEDTAVYYCARDNYGSSGKFFAYWGQGTLVTVSS

SEQ ID NO: 20 - protein, 107 aa, synthetic construct [sequence omitted]

SEQ ID NO: 21 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK